SEQUENCE LISTING (2 sequences)

SEQ ID NO: 1 - DNA, 2616 bp, human
atggcggattccagcgaaggcccccgcgcggggcccggggaggtggctgagctccccggggatgagagtggcaccccaggtggggaggcttttcctctctcctccctggccaatctgtttgagggggaggatggctccctttcgccctcaccggctgatgccagtcgccctgctggcccaggcgatgggcgaccaaatctgcgcatgaagttccagggcgccttccgcaagggggtgcccaaccccatcgatctgctggagtccaccctatatgagtcctcggtggtgcctgggcccaagaaagcacccatggactcactgtttgactacggcacctatcgtcaccactccagtgacaacaagaggtggaggaagaagatcatagagaagcagccgcagagccccaaagcccctgcccctcagccgccccccatcctcaaagtcttcaaccggcctatcctctttgacatcgtgtcccggggctccactgctgacctggacgggctgctcccattcttgctgacccacaagaaacgcctaactgatgaggagtttcgagagccatctacggggaagacctgcctgcccaaggccttgctgaacctgagcaatggccgcaacgacaccatccctgtgctgctggacatcgcggagcgcaccggcaacatgcgggagttcattaactcgcccttccgtgacatctactatcgaggtcagacagccctgcacatcgccattgagcgtcgctgcaaacactacgtggaacttctcgtggcccagggagctgatgtccacgcccaggcccgtgggcgcttcttccagcccaaggatgaggggggctacttctactttggggagctgcccctgtcgctggctgcctgcaccaaccagccccacattgtcaactacctgacggagaacccccacaagaaggcggacatgcggcgccaggactcgcgaggcaacacagtgctgcatgcgctggtggccattgctgacaacacccgtgagaacaccaagtttgttaccaagatgtacgacctgctgctgctcaagtgtgcccgcctcttccccgacagcaacctggaggccgtgctcaacaacgacggcctctcgcccctcatgatggctgccaagacgggcaagattgggatctttcagcacatcatccggcgggaggtgacggatgaggacacacggcacctgtcccgcaagttcaaggactgggcctatgggccagtgtattcctcgctttatgacctctcctccctggacacgtgtggggaagaggcctccgtgctggagatcctggtgtacaacagcaagattgagaaccgccacgagatgctggctgtggagcccatcaatgaactgctgcgggacaagtggcgcaagttcggggccgtctccttctacatcaacgtggtctcctacctgtgtgccatggtcatcttcactctcaccgcctactaccagccgctggagggcacaccgccgtacccttaccgcaccacggtggactacctgcggctggctggcgaggtcattacgctcttcactggggtcctgttcttcttcaccaacatcaaagacttgttcatgaagaaatgccctggagtgaattctctcttcattgatggctccttccagctgctctacttcatctactctgtcctggtgatcgtctcagcagccctctacctggcagggatcgaggcctacctggccgtgatggtctttgccctggtcctgggctggatgaatgccctttacttcacccgtgggctgaagctgacggggacctatagcatcatgatccagaagattctcttcaaggaccttttccgattcctgctcgtctacttgctcttcatgatcggctacgcttcagccctggtctccctcctgaacccgtgtgccaacatgaaggtgtgcaatgaggaccagaccaactgcacagtgcccacttacccctcgtgccgtgacagcgagaccttcagcaccttcctcctggacctgtttaagctgaccattggcatgggcgacctggagatgctgagcagcaccaagtaccccgtggtcttcatcatcctgctggtgacctacatcatcctcacctttgtgctgctcctcaacatgctcattgccctcatgggcgagacagtgggccaggtctccaaggagagcaagcacatctggaagctgcagtgggccaccaccatcctggacattgagcgctccttccccgtattcctgaggaaggccttccgctctggggagatggtcaccgtgggcaagagctcggacggcactcctgaccgcaggtggtgcttcagggtggatgaggtgaactggtctcactggaaccagaacttgggcatcatcaacgaggacccgggcaagaatgagacctaccagtattatggcttctcgcataccgtgggccgcctccgcagggatcgctggtcctcggtggtaccccgcgtggtggaactgaacaagaactcgaacccggacgaggtggtggtgcctctggacagcatggggaacccccgctgcgatggccaccagcagggttacccccgcaagtggaggactgatgacgccccgctctag

SEQ ID NO: 2 - protein, 871 aa, human
MADSSEGPRAGPGEVAELPGDESGTPGGEAFPLSSLANLFEGEDGSLSPSPADASRPAGPGDGRPNLRMKFQGAFRKGVPNPIDLLESTLYESSVVPGPKKAPMDSLFDYGTYRHHSSDNKRWRKKIIEKQPQSPKAPAPQPPPILKVFNRPILFDIVSRGSTADLDGLLPFLLTHKKRLTDEEFREPSTGKTCLPKALLNLSNGRNDTIPVLLDIAERTGNMREFINSPFRDIYYRGQTALHIAIERRCKHYVELLVAQGADVHAQARGRFFQPKDEGGYFYFGELPLSLAACTNQPHIVNYLTENPHKKADMRRQDSRGNTVLHALVAIADNTRENTKFVTKMYDLLLLKCARLFPDSNLEAVLNNDGLSPLMMAAKTGKIGIFQHIIRREVTDEDTRHLSRKFKDWAYGPVYSSLYDLSSLDTCGEEASVLEILVYNSKIENRHEMLAVEPINELLRDKWRKFGAVSFYINVVSYLCAMVIFTLTAYYQPLEGTPPYPYRTTVDYLRLAGEVITLFTGVLFFFTNIKDLFMKKCPGVNSLFIDGSFQLLYFIYSVLVIVSAALYLAGIEAYLAVMVFALVLGWMNALYFTRGLKLTGTYSIMIQKILFKDLFRFLLVYLLFMIGYASALVSLLNPCANMKVCNEDQTNCTVPTYPSCRDSETFSTFLLDLFKLTIGMGDLEMLSSTKYPVVFIILLVTYIILTFVLLLNMLIALMGETVGQVSKESKHIWKLQWATTILDIERSFPVFLRKAFRSGEMVTVGKSSDGTPDRRWCFRVDEVNWSHWNQNLGIINEDPGKNETYQYYGFSHTVGRLRRDRWSSVVPRVVELNKNSNPDEVVVPLDSMGNPRCDGHQQGYPRKWRTDDAPL

(19) United States
(12) Patent Application Publication
(10) Pub. No.: US 2007/0259856 A1
(43) Pub. Date: Nov. 8, 2007

(54) Method for Activating Trpv4 Channel Receptors by Agonists

(76) Inventors: Sanjay Kumar, Collegeville, PA (US); Michael A. Pratta, Collegeville, PA (US); Bartholomew Jude Votta, Collgeville, PA (US)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(21) Appl. No.: 11/574,749

(22) PCT Filed: Sep. 7, 2005
(86) PCT No.: PCT/US05/31872

§ 371 Date: Mar. 6, 2007

Related U.S. Application Data
(60) Provisional application No. 60/607,544, filed on Sep. 7, 2004.

(51) Int. Cl. A61K 31/135; A61K 31/55; A61P 19/02

(52) U.S. Cl. 514/217.11; 514/443

(57) ABSTRACT

This invention relates to methods for activating a TRPV4 channel receptor, thereby reducing the production and/or release of matrix degrading enzymes by a cell expressing a TRPV4 channel receptor, thereby reducing the breakdown of an extra-cellular matrix. Also contemplated within the scope of the invention are methods of attenuating the inhibition of matrix production.

### FIELD OF THE INVENTION

This invention relates to methods for activating a TRPV4 channel receptor, thereby reducing the production and/or release of matrix degrading enzymes by cells expressing a TRPV4 channel receptor, thereby reducing the breakdown of extracellular matrix. Also contemplated within the scope of the invention are methods of attenuating inhibition of matrix production.

### BACKGROUND OF THE INVENTION

Cartilage is an avascular tissue populated by specialized cells termed chondrocytes, which respond to diverse mechanical and biochemical stimuli. Cartilage is present in the linings of joints, interstitial connective tissues, and basement membranes, and is composed of an extracellular matrix comprised of several matrix components including type II collagen, proteoglycans, fibronectin and laminin.

In normal cartilage, extracellular matrix synthesis is offset by extracellular matrix degradation, resulting in normal matrix turnover. Depending on the signal(s) received, the ensuing response may be either anabolic (leading to matrix production and/or repair) or catabolic (leading to matrix degradation, cellular apoptosis, loss of function, and pain).

In response to injurious compression and/or exposure to inflammatory mediators (e.g. inflammatory cytokines) chondrocytes decrease matrix production and increase production of multiple matrix degrading enzymes. Examples of matrix degrading enzymes include aggrecanases (ADAMTSs) and matrix metalloproteases (MMPs). The activities of these enzymes result in the degradation of the cartilage matrix. Aggrecanases (ADAMTSs), in conjunction with MMPs, degrade aggrecan, an aggregating proteoglycan present in articular cartilage. In osteoarthritic (OA) articular cartilage a loss of proteoglycan staining is observed in the superficial zone in early OA and adjacent to areas of cartilage erosion in moderate to severe OA. The reduction in proteoglycan content is associated with an increase in degradation of type II collagen by specialized MMPs, termed collagenases (e.g. MMP-13). Collagenases are believed to make the initial cleavage within the triple-helix of intact collagen. It's hypothesized that the initial cleavage of collagen by collagenases facilitates the further degradation of the collagen fibrils by other proteases. Thus, preventing or reducing the increased production of matrix degrading enzymes and/or attenuating the inhibition of matrix production may also promote functional recovery.

Excessive degradation of extracellular matrix is implicated in the pathogenesis of many diseases and conditions, including pain, chronic pain, neuropathic pain, postoperative pain, rheumatoid arthritis, osteoarthritis, neuralgia, neuropathies, algesia, nerve injury, ischaemia, neurodegeneration, cartilage degeneration, stroke, incontinence, inflammatory disorders, irritable bowel syndrome, periodontal disease, aberrant angiogenesis, tumor invasion and metastasis, corneal ulceration, and in complications of diabetes.

TRPV4 channel receptor is one of six known members of the vanilloid family of transient receptor potential channels and shares 51% identity at the nucleotide level with TRPV1, the capsaicin receptor. Examples of polypeptides and polynucleotides encoding forms of human vanniloid receptors, including TRPV4 channel receptor from human, can be found in EP 1170365 as well as WO 00/32766. A polypeptide sequence of TRPV4 channel receptor is presented in SEQ ID NO: 1 (See FIG. 2), and a polynucleotide sequence encoding human TRPV4 receptor is presented in SEQ ID NO: 2 (See FIG. 3) herein. Like the other family members, TRPV4 channel receptor is a Ca2+ permeable, non-selective, ligand-gated cation channel, which is responsive to diverse stimuli such as reduced osmolality, elevated temperature, and small molecule ligands. See, for instance, Voets, et al., J. Biol. Chem. (2002) 277 33704-47051; Watanabe, et al., J. Biol. Chem. (2002) 277:47044-47051; Watanabe, et al., J. Biol. Chem. (2002) 277: 13569-47051; Xu, et al., J. Biol. Chem. (2003) 278:11520-11527. From a screen of body tissues, the human TRPV4 channel receptor is most prominently expressed in cartilage. A screen of primary and clonal cell cultures shows significant expression only in chondrocytes. Phorbol 12-myristate 13-acetate (PMA) and 4α-phorbol-12,13-didecanoate (4αPDD) have been shown to increase intracellular calcium in cells transfected with vectors encoding TRPV4 channel receptor (WO 02/34280). The present invention demonstrates herein that modulation of a TRPV4 channel receptor can attenuate matrix degrading enzyme production as well as attenuate inhibition of matrix production.

Accordingly, a method for modulating the activity of a TRPV4 channel receptor causing an attenuation of the aforementioned catabolic responses is greatly needed and would provide a novel therapeutic approach for the treatment of diseases involving matrix degradation.

### SUMMARY OF THE INVENTION

The present invention provides a method for activating a TRPV4 channel receptor or a TRPV4 channel receptor variant in at least one cell expressing a TRPV4 channel receptor or a TRPV4 channel receptor variant comprising contacting at least one cell with an effective amount of pharmaceutical composition comprising an agonist to the said TRPV4 channel receptor. In one aspect, at least one cell is from a human. In another aspect, at least one cell is a chondrocyte. In another aspect, at least one cell forms part of a cartilage matrix.

In another aspect, the agonist reduces the amount of matrix degrading enzymes produced by at least one cell. In another aspect, the agonist reduces the amount of matrix degrading enzymes released by at least one cell. In another aspect, the agonist reduces the amount of aggrecanase produced by at least one cell. In another aspect, the agonist reduces the amount of aggrecanases released by at least one cell. In another aspect, the agonist reduces the amount of matrix metalloproteases (“MMPs”) produced by at least one cell. In another aspect, the agonist reduces the amount of MMPs released by at least one cell. In another aspect, the MMPs are chosen from, but are not limited to, the group: MMP-1, MMP-3 and MMP-13.

In another aspect, the agonist reduces the amount of nitric oxide produced by at least one cell. In another aspect, the agonist reduces the amount of nitric oxide released by at least one cell. In another aspect, the agonist attenuates inhibition of proteoglycan synthesis. In another aspect, a method is provided for increasing current flow through a TRPV4 channel receptor.

In another aspect, agonist comprises a first chemical moiety selected from the group of: aryl or heteroaryl and a second chemical moiety which is an aryl optionally substituted with CN, NO₂, halogen, and wherein the first and second chemical moiety form part of the same compound and are connected through a covalent chemical structure. The first chemical moiety may be selected from the group of: benzothiophene, indenyl or indole and a second chemical moiety may be selected from the group of: halogenated benzene or cyano-benzene.

In another aspect, the agonist comprises 3-oxohexahydro-1H-azepin or azepines. In another aspect, the agonist is chosen from the group of: 3-oxohexahydro-1H-azepin and azepines.

In another aspect, the agonist is not phorbol 12-myristate 13-acetate (PMA) or 4α-phorbol-12,13-didecanoate (4α-PDD). In another aspect, the agonist comprises a 1,3-diamine. In another aspect, the agonist comprises an optionally substituted benzylsulfonamide. In another aspect, the agonist comprises a first chemical moiety selected from the group of: aryl or heteroaryl and a second chemical moiety which is an aryl optionally substituted with CN, NO₂, halogen, and wherein the first and second chemical moiety form part of the same compound. The agonist may further comprise a 1,3-diamine. In another aspect, the agonist comprises a first chemical moiety selected from the group of: benzothiophene, indenyl or indole and a second chemical moiety selected from the group of: halogenated benzene or cyano-benzene.

In another aspect, the agonist is chosen from the group of: N-{(1S)-1-[({(4R)-1-[(4-chlorophenyl)sulfonyl]-3-oxohexahydro-1H-azepin-4-yl}amino)carbonyl]-3-methylbutyl}-1-benzothiophene-2-carboxamide; N-{(1S)-1-[({(4R)-1-[(4-fluorophenyl)sulfonyl]-3-oxohexahydro-1H-azepin-4-yl}amino)carbonyl]-3-methylbutyl}-1-benzothiophene-2-carboxamide; N-{(1S)-1-[({(4R)-1-[(2-cyanophenyl)sulfonyl]-3-oxohexahydro-1H-azepin-4-yl}amino)carbonyl]-3-methylbutyl}-1-methyl-1H-indole-2-carboxamide; N-{(1S)-1-[({(4R)-1-[(2-cyanophenyl)sulfonyl]hexahydro-1H-azepin-4-yl}amino)carbonyl]-3-methylbutyl}-1-methyl-1H-indole-2-carboxamide; N-{(1S)-1-[({3-[[(cyanophenyl)sulfonyl](methyl)amino]propyl}amino)carbonyl]-3-methylbutyl}-1-benzothiophene-2-carboxamide; and N-{(1S)-1-[({3-[[(2,4-dichlorophenyl)sulfonyl](methyl)amino]propyl}amino)carbonyl]-3-methylbutyl}-1-benzothiophene-2-carboxamide.

In another aspect, the agonist has an EC50 value for TRPV4 channel receptor of less than about 1.0 μM. In another aspect, the agonist has an EC50 value for TRPV4 channel receptor of less than about 10 nM. In another aspect, the agonist inhibits GAG release induced by catabolic stimuli in articular cartilage explants with an IC50 value of less than about 1.0 μM. In another aspect, the agonist has an EC50 value for TRPV4 channel receptor of less than about 1.0 μM as measured by calcium influx in isolated chondrocytes. In another aspect, the agonist increases current flow through said TRPV4 channel receptor.

In another embodiment, the present invention provides a method for treating a patient in need thereof comprising contacting at least one cell expressing a TRPV4 channel receptor of the patient with a therapeutically effective amount of an agonist to a TRPV4 channel receptor. In one aspect, the patient is suffering from a disease or condition of the cartilage. In another aspect, the patient is suffering from a disease or condition chosen from the group of: pain, chronic pain, neuropathic pain, postoperative pain, osteoarthritis, neuralgia, neuropathies, neurodegeneration, cartilage degeneration, periodontal disease, aberrant angiogenesis, corneal ulceration, and complications of diabetes. In another aspect, the patient suffers from a disease affecting the larynx, trachea, auditory canal, intervertebral discs, ligaments, tendons, joint capsules or bone development. In another aspect, the disease is related to joint destruction. In another aspect, the patient is suffering from osteoarthritis. In another aspect, the patient is suffering from rheumatoid arthritis.

In another aspect, the agonist comprises 3-oxohexahydro-1H-azepin, azepine or acyclic 1,3-diamine. In another aspect, the agonist is chosen from the group of: 3-oxohexahydro-1H-azepin and azepines. In another aspect, the agonist is chosen from the group of: 3-oxohexahydro-1H-azepin, azepine and acyclic 1,3-diamine.

In another aspect, treatment of the patient reduces the amount of aggrecan degradation in the patient. In another aspect, treatment of the patient reduces the amount of collagen degradation in the patient. In another aspect, treatment of the patient attenuates cartilage degradation, which may be in response to inflammatory mediators. In another aspect, cartilage degradation in the patient may be in response to injury.

In another aspect, a method is provided for attenuating decreased matrix protein production in the patient. In another aspect, the matrix protein is chosen from the group of: aggrecan, type II collagen, and type VI collagen. In another aspect, a method is provided for attenuating increased production of matrix degrading enzymes. In another aspect, the matrix degrading enzymes are chosen from, but are not limited to, the group of: MMP-1, MMP-3, MMP-9, MMP-13, ADAMTS4, and ADAMTS5. In another aspect, the production of matrix degrading enzymes is induced by inflammatory mediators. In another aspect, the production of matrix degrading enzymes is induced due to injury. In another aspect, a method is provided for reducing the amount of nitric oxide produced by at least one cell in cartilage. In another aspect, a method is provided for attenuating inhibition of proteoglycan synthesis.

In another aspect of the present invention, compounds are provided comprising a 1,3-diamine wherein a compound activates a TRPV4 channel receptor or a TRPV4 channel receptor variant when said compound is contacted with at least one cell expressing said TRPV4 channel receptor or said TRPV4 channel receptor variant. In another aspect, the compound comprises an optionally substituted benzylsulfonamide. In yet another aspect, the compound comprises a first chemical moiety selected from the group of: aryl or heteroaryl and a second chemical moiety which is an aryl optionally substituted with CN, NO₂, halogen, and wherein the first and second chemical moiety are comprised in the same compound. In another aspect, the compound comprises a first chemical moiety selected from the group of: benzothiophene, indenyl, or indole and a second chemical moiety selected from the group of: halogenated benzene or cyano-benzene.

In yet another aspect of the present invention, compounds are provided such that when a compound is contacted with at least one cell expressing at least one TRPV4 channel receptor or at least one TRPV4 channel receptor variant, the compound reduces an amount of at least one type of matrix degrading enzymes produced by said at least one cell. In another aspect, the compound reduces an amount of at least one type of matrix degrading enzymes produced or released by at least one cell expressing at least one TRPV4 channel receptor or at least one TRPV4 channel receptor variant. The compound may also reduce the amount of aggrecanase produced or released by a cell expressing at least one TRPV4 channel receptor or at least one TRPV4 channel receptor variant. Also provided are compounds that reduce an amount of at least one type of matrix metalloproteases produced or released by said at least one cell. Compounds of the present invention may also reduce the amount of nitric oxide produced or released by a cell expressing at least one TRPV4 channel receptor or at least one TRPV4 channel receptor variant. In another aspect, compound of the present invention may attenuate inhibition of proteoglycan synthesis in a cell expressing at least one TRPV4 channel receptor or at least one TRPV4 channel receptor variant.

### BRIEF DESCRIPTION OF THE DRAWINGS

FIG. 1: Whole cell patch clamp measurements showing the voltage- and time-dependent effects of Formula IId on TRPV4 channel receptor-mediated current in HEK293 MSRII cells transiently transduced with 5% hVR4 BacMam virus.

FIG. 2: Polynucleotide sequence encoding human TRPV4.

FIG. 3: Polypeptide sequence encoded by polynucleotide sequence of FIG. 2.

### DETAILED DESCRIPTION OF THE INVENTION

“Polynucleotide” generally refers to any polyribonucleotide or polydeoxyribonucleotide, which may be unmodified RNA or DNA or modified RNA or DNA. “Polynucleotides” include, without limitation single- and double-stranded DNA, DNA that is a mixture of single- and double-stranded regions, single- and double-stranded RNA, and RNA that is mixture of single- and double-stranded regions, hybrid molecules comprising DNA and RNA that may be single-stranded or, more typically, double-stranded or a mixture of single- and double-stranded regions. In addition, “polynucleotide” refers to triple-stranded regions comprising RNA or DNA or both RNA and DNA. The term polynucleotide also includes DNAs or RNAs containing one or more modified bases and DNAs or RNAs with backbones modified for stability or for other reasons. “Modified” bases include, for example, tritylated bases and unusual bases such as inosine. A variety of modifications has been made to DNA and RNA; thus, “polynucleotide” embraces chemically, enzymatically or metabolically modified forms of polynucleotides as typically found in nature, as well as the chemical forms of DNA and RNA characteristic of viruses and cells. “Polynucleotide” also embraces relatively short polynucleotides, often referred to as oligonucleotides.

“Polypeptide” refers to any peptide or protein comprising two or more amino acids joined to each other by peptide bonds or modified peptide bonds, i.e., peptide isosteres. “Polypeptide” refers to both short chains, commonly referred to as peptides, oligopeptides or oligomers, and to longer chains, generally referred to as proteins. Polypeptides may contain amino acids other than the 20 gene-encoded amino acids. “Polypeptides” include amino acid sequences modified either by natural processes, such as posttranslational processing, or by chemical modification techniques that are well known in the art. Such modifications are well described in basic texts and in more detailed monographs, as well as in a voluminous research literature. Modifications can occur anywhere in a polypeptide, including the peptide backbone, the amino acid side-chains and the amino or carboxyl termini. It will be appreciated that the same type of modification may be present in the same or varying degrees at several sites in a given polypeptide. Also, a given polypeptide may contain many types of modifications. Polypeptides may be branched as a result of ubiquitination, and they may be cyclic, with or without branching. Cyclic, branched and branched cyclic polypeptides may result from posttranslation natural processes or may be made by synthetic methods. Modifications include acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cysteine, formation of pyroglutamate, formylation, gamma-carboxylation, glycosylation, GPI anchor formation, hydroxylation, iodination, methylation, myristoylation, oxidation, proteolytic processing, phosphorylation, prenylation, racemization, selenoylation, sulfation, transfer-RNA mediated addition of amino acids to proteins such as arginylation, and ubiquitination. See, for instance, PROTEINS—STRUCTURE AND MOLECULAR PROPERTIES, 2nd Ed., T. E. Creighton, W. H. Freeman and Company, New York, 1993 and Wold, F., Posttranslational Protein Modifications: Perspectives and Prospects, pgs. 1-12 in POSTTRANSLATIONAL COVALENT MODIFICATION OF PROTEINS, B. C. Johnson, Ed., Academic Press, New York, 1983; Seifter, et al., “Analysis for protein modifications and nonprotein cofactors”, Meth. Enzymol. (1990) 182:626-646 and Rattan, et al., “Protein Synthesis: Posttranslational Modifications and Aging”, Ann NY Acad Sci (1992) 663:48-62.

“Variant” as the term is used herein, is a polynucleotide or polypeptide that differs from a reference polynucleotide or polypeptide respectively, but retains essential properties. A typical variant of a polynucleotide differs in nucleotide sequence from another, reference polynucleotide. Changes in the nucleotide sequence of the variant may or may not alter the amino acid sequence of a polypeptide encoded by the reference polynucleotide. Nucleotide changes may result in amino acid substitutions, additions, deletions, fusions and truncations in the polypeptide encoded by the reference sequence, as discussed below. A typical variant of a polypeptide differs in amino acid sequence from another, reference polypeptide. Generally, differences are limited so that the sequences of the reference polypeptide and the variant are closely similar overall and, in many regions, identical. A variant and reference polypeptide may differ in amino acid sequence by one or more substitutions, additions, deletions in any combination. A substituted or inserted amino acid residue may or may not be one encoded by the genetic code. A variant of a polynucleotide or polypeptide may be a naturally occurring such as an allelic variant, or it may be a variant that is not known to occur naturally. Non-naturally occurring variants of polynucleotides and polypeptides may be made by mutagenesis techniques or by direct synthesis.

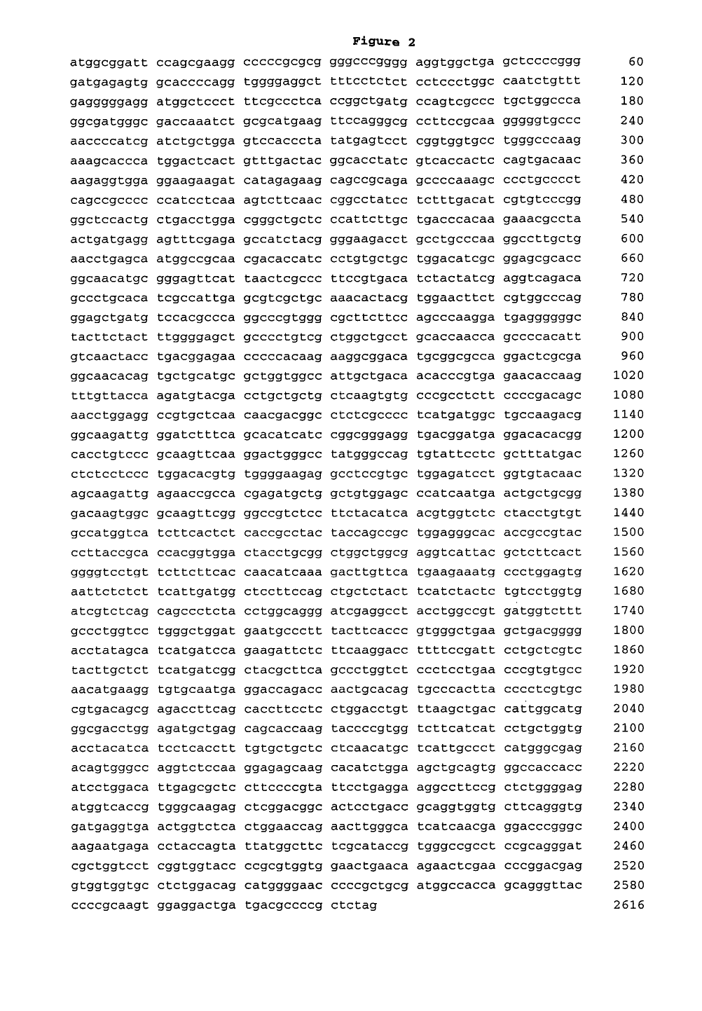

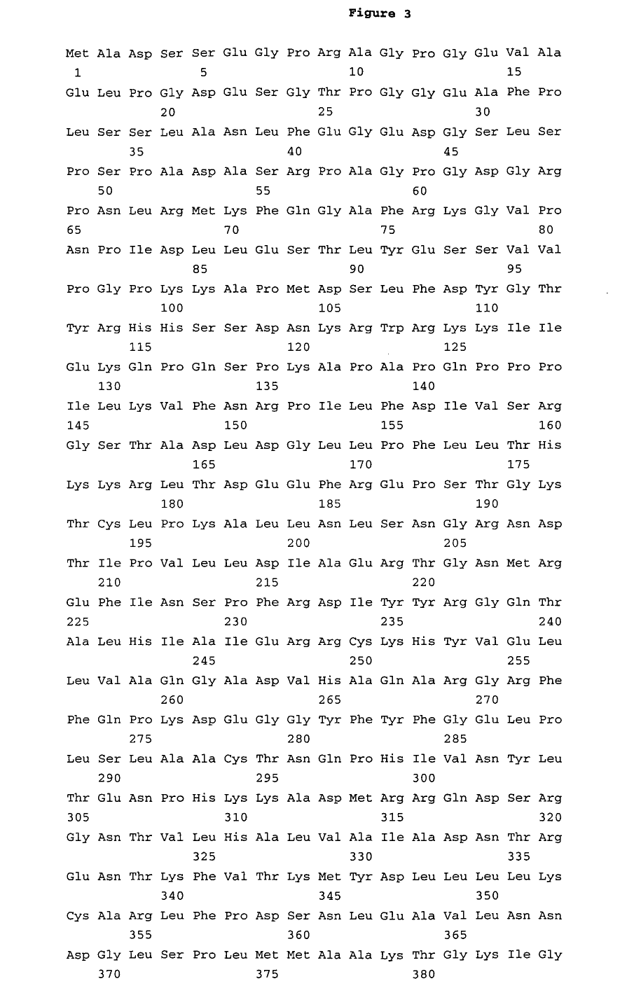

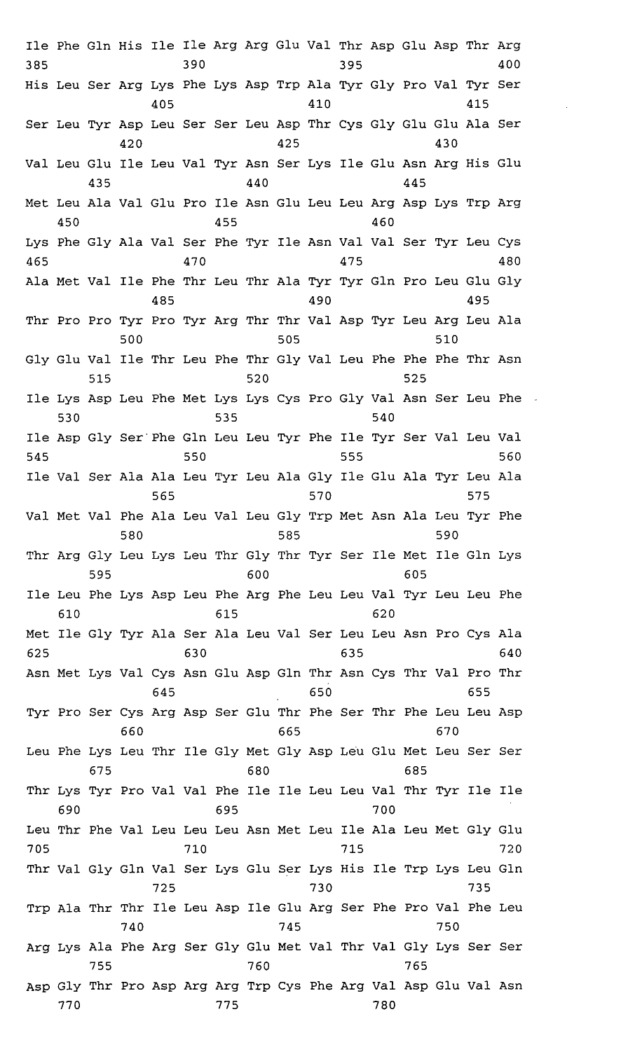

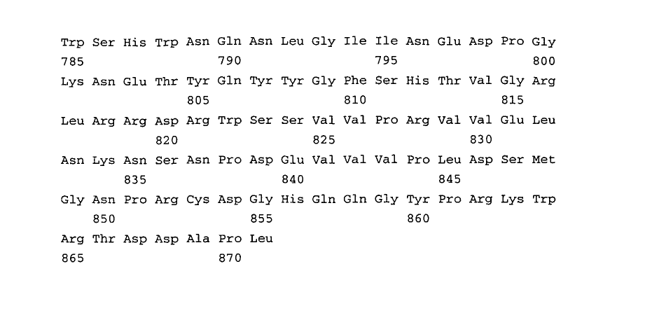

As used herein “TRPV4 channel receptor” refers to one of six known members of the vanilloid family of transient receptor potential channels and shares 51% identity at the nucleotide level with TRPV1, the capsaicin receptor. Examples of polypeptides and polynucleotides encoding forms of human vanniloid receptors, including TRPV4 channel receptor from human, can be found in EP 1170365 as well as WO 00/32766. A polypeptide sequence of TRPV4 channel receptor is presented in SEQ ID NO: 1 (See FIG. 2), and a polynucleotide sequence encoding human TRPV4 receptor is presented in SEQ ID NO: 2 (See FIG. 3) herein.

As used herein “TRPV4 channel receptor variant” refers a polynucleotide or polypeptide that differs from a TRPV4 channel receptor encoding polynucleotide or polypeptide respectively, but retains essential properties. TRPV4 channel receptor variants may vary from a TRPV4 channel receptor polypeptide by insertions, deletions, and substitutions that may be conservative or non-conservative, or any combination thereof. Particularly preferred variants are those in which several, for instance from 50 to 30, from 30 to 20, from 20 to 10, from 10 to 5, from 5 to 3, from 3 to 2, from 2 to 1 or 1 amino acids are inserted, substituted, or deleted, in any combination. TRPV4 channel receptor variants of a TRPV4 encoding polynucleotide may have about 99%, 97%, 95%, 90%, 85% or 80% sequence identity with SEQ ID NO: 1. TRPV4 channel receptor variants of a TRPV4 polypeptide may have about 99%, 97%, 95%, 90%, 85% or 80% sequence identity with SEQ ID NO:2.

As used herein “agonist” to a TRPV4 channel receptor includes any compound capable of activating or enhancing the biological activities of a TRPV4 channel receptor.

As used herein “activating” a TRPV4 channel receptor may include, but is not limited to, such outcomes as increasing current flow through a TRPV4 channel by increasing its mean open time and/or its open probability, inducing an influx of calcium (or other monovalent and/or divalent cations) into the cell, reducing the amount of ADAMTSs produced and/or released by the cell, reducing the amount of MMPs produced and/or released by the cell, inhibiting the basal or growth factor-stimulated proliferation of the cell, reducing the amount of nitric oxide (NO) produced by a cell, and attenuating the inhibition of matrix synthesis.

As used herein “inflammatory mediators” include any compound capable of triggering an inflammatory process. The term inflammation generally refers to the process of reaction of vascularized living tissue to injury. This process includes but is not limited to increased blood flow, increased vascular permeability, and leukocytic exudation. Because leukocytes recruited into inflammatory reactions can release potent enzymes and oxygen free radicals (i.e. inflammatory mediators), the inflammatory response is capable of mediating considerable tissue damage. Examples of inflammatory mediators include, but are not limited to prostaglandins (e.g. PGE2), leukotrienes (e.g. LTB4), inflammatory cytokines, such as tumour necrosis factor alpha (TNFα), interleukin 1 (IL-1), and interleukin 6 (IL-6); nitric oxide (NO), metalloproteinases, and heat shock proteins.

As used herein “matrix protein” includes proteins released from cells to form the extracellular matrix of cartilage. The extracellular matrix of cartilage consists of proteoglycans, belonging to several distinct proteoglycan families. These include, but are not limited to, perlecan and the hyalectans, exemplified by aggrecan and versican, and the small leucine-rich family of proteoglycans, including decorin, biglycan and fibromodulin. The extracellular matrix also consists of hybrid collagen fibers comprised of three collagen isotypes, namely type II, type VI, type IX, and type XI collagens, along with accessory proteins such as cartilage oligeromeric matrix protein (COMP), link protein, and fibronectin. Cartilage also contains hyaluronin which forms a noncovalent association with the hyalectins. In addition, a specialized pericellular matrix surrounds the chondrocyte which consists of proteoglycans, type VI collagen and collagen receptor proteins, such as anchorin.

As used herein “matrix degrading enzymes” refers to enzymes able to cleave extracellular matrix proteins. Cartilage extracellular matrix turnover is regulated by matrix metalloproteases (MMPs) which are synthesized as latent proenzymes that require activation in order to degrade cartilage extracellular matrix proteins. Three classes of enzymes are believed to regulate the turnover of extracellular matrix proteins, namely collagenases (including, but not limited to, MMP-13), responsible for the degradation of native collagen fibers, stromelysins (including, but not limited to, MMP-3) which degrade proteoglycan and type IX collagen, and gelatinases (including, but not limited to, MMP-2 and MMP-9) which degrade denatured collagen. The matrix degrading enzyme group that appears most relevant in cartilage degradation in OA includes a subgroup of metalloproteinases called ADAMTS, because they possess disintegrin and metalloproteinase domains and a thrombospondin motif in their structure. ADAMTS4 (aggrecanase-1) has been reported to be elevated in OA joints and along with ADAMTS-5 (aggrecanase-2) have been shown to be expressed in human osteoarthritic cartilage. These enzymes appear to be responsible for aggrecan degradation without MMP participation. Thus, an inhibition of activity or a reduction in expression of these enzymes may have utility in OA therapy. Stanton, et al., Nature 434:648-652 (31 Mar. 2005).

As used herein, “reduce” or “reducing” the production of matrix degrading enzymes refers to a decrease in the amount of matrix degrading enzyme(s) produced and/or released by a cell, which has exhibited an increase in matrix degrading enzyme production or release in response to a catabolic stimulus, which may include, but is not limited to, physical injury, mechanical and/or osmotic stress, or exposure to an inflammatory mediator.

As used herein “attenuate” or “attenuating” refers to a normalization (i.e., either an increase or decrease) of the amount of matrix degrading enzyme, inflammatory mediator, or matrix protein produced and/or released by a cell, following exposure to a catabolic stimulus. For example, following exposure to IL-1 chondrocyte production of matrix proteins, such as proteoglycans, are reduced, while production of matrix degrading enzymes (e.g. MMP-13, ADAMTS4) and reactive oxygen species (e.g. NO) are increased. Attenuation refers to the normalization of these diverse responses to levels observed in the absence of a catabolic stimulus.

As used herein and as is understood in the art “EC50” or “effective concentration 50%” refers to the molar concentration of an agonist, which produces 50% of the maximum possible stimulatory response for that agonist. The maximum stimulatory response for each agonist is determined experimentally by measuring the magnitude of the desired biological response elicited by increasing concentrations of agonist until a plateau is achieved.

As used herein and as is understood in the art “IC50” or “inhibitory concentration 50%” refers to the molar concentration of a compound (agonist, antagonist, or inhibitor) which produces 50% of the maximum possible inhibitory response for that compound. The maximum inhibitory response for each compound is determined experimentally by measuring the extent of inhibition of the desired biological response elicited by increasing concentrations of agonist until a plateau is achieved.

As used herein “Aryl” or “Ar” means phenyl or naphthyl. Aryl groups may be optionally substituted with one or more substituents as defined herein. Aryl groups may be optionally substituted with up to five groups selected from (C₁₋₄)alkylthio; halo; carboxy(C₁₋₄)alkyl; halo(C₁₋₄)alkoxy; halo(C₁₋₄)alkyl; (C₁₋₄)alkyl; (C₂₋₄)alkenyl; (C₁₋₄)alkoxycarbonyl; formyl; (C₁₋₄)alkylcarbonyl; (C₂₋₄)alkenyloxycarbonyl; (C₂₋₄)alkenylcarbonyl; (C₁₋₄)alkylcarbonyloxy; (C₁₋₄)alkoxycarbonyl(C₁₋₄)alkyl; hydroxy; hydroxy(C₁₋₄)alkyl; mercapto(C₁₋₄)alkyl; (C₁₋₄)alkoxy; nitro; cyano; carboxy; amino or aminocarbonyl; (C₁₋₄)alkylsulphonyl; (C₂₋₄)alkenylsulphonyl; or aminosulphonyl wherein the amino group is optionally substituted by (C₁₋₄)alkyl or (C₂₋₄)alkenyl; phenyl, phenyl(C₁₋₄)alkyl or phenyl(C₁₋₄)alkoxy.

As used herein “Enantiomerically enriched” refers to products whose enantiomeric excess is greater than zero. For example, enantiomerically enriched refers to products whose enantiomeric excess is greater than about 50% ee, greater than about 75% ee, and greater than about 90% ee.

As used herein “Enantiomeric excess” or “ee” is the excess of one enantiomer over the other expressed as a percentage. As a result, since both enantiomers are present in equal amounts in a racemic mixture, the enantiomeric excess is zero (0% ee). However, if one enantiomer was enriched such that is constitutes 95% of the product, then the enantiomeric excess would be 90% ee (the amount of the enriched enantiomer, 95%, minus the amount of the other enantiomer, 5%).

As used herein “Enantiomerically pure” refers to products whose enantiomeric excess is 100% ee.

As used herein “Diasteriomer” refers to a compound having at least two chiral centers.

As used herein “Diasteriomer excess” or “de” is the excess of one diasteriomer over the others expressed as a percentage.

As used herein “Diasteriomerically pure” refers to products whose diasteriomeric excess is 100% de.

“Heteroaryl” refers to an aromatic ring containing from 1 to 4 heteroatoms as member atoms in the ring. Heteroaryl groups containing more than one heteroatom may contain different heteroatoms. Heteroaryl groups may be optionally substituted with one or more substituents as defined herein. Heteroaryl groups are monocyclic ring systems or are fused, spiro, or bridged bicyclic ring systems. Monocyclic heteroaryl rings have from 5 to 7 member atoms. Bicyclic heteroaryl rings have from 7 to 11 member atoms. Bicyclic heteroaryl rings include those rings wherein phenyl and a monocyclic heterocycloalkyl ring are attached forming a fused, spiro, or bridged bicyclic ring system, and those rings wherein a monocyclic heteroaryl ring and a monocyclic cycloalkyl, cycloalkenyl, heterocycloalkyl, or heteroaryl ring are attached forming a fused, spiro, or bridged bicyclic ring system. Heteroaryl includes, but is not limited to, pyrrolyl, pyrazolyl, imidazolyl, oxazolyl, isoxazolyl, thiazolyl, isothiazolyl, furanyl, furazanyl, thienyl, triazolyl, tetrahydrofuranyl, pyridinyl, pyrimidinyl, pyridazinyl, pyrazinyl, triazinyl, tetrazinyl, indolyl, isoindolyl, indolizinyl, indazolyl, purinyl, quinolinyl, isoquinolinyl, quinoxalinyl, quinazolinyl, pteridinyl, cinnolinyl, benzimidazolyl, benopyranyl, benzoxazolyl, benzofuranyl, isobenzofuranyl, benzothiazolyl, benzothienyl, furopyridinyl, and napthyridinyl.

As used herein “amino acid” refers to the D- or L-isomers of alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine and valine.

As used here “amide bond” refers to an amide linkage formed between a carboxylic acid and an amine. An amide bond typically has a distance of approximately 1.23 Angstroms between the carbonyl carbon atom and the adjacent nitrogen atom.

“Alkyl” refers to a saturated hydrocarbon chain having from 1 to 12 member atoms. Alkyl groups may be optionally substituted with one or more substituents as defined herein. Use of the prefix “C1-x” or “C1-Cx” with alkyl refers to an alkyl group having from 1 to x member atoms. For example, C₁₋₆alkyl refers to an alkyl group having from 1 to 6 member atoms. Alkyl groups may be straight or branched. Representative branched alkyl groups have one, two, or three branches. Alkyl includes methyl, ethyl, propyl (n-propyl and isopropyl), butyl (n-butyl, isobutyl, and t-butyl), pentyl (n-pentyl, isopentyl, and neopentyl), and hexyl. Unless otherwise defined, the term C₁₋₆alkyl (or alternatively as (C₁₋₆)alkyl) when used alone or when forming part of other groups (such as the ‘alkoxy’ group) includes substituted or unsubstituted, straight or branched chain alkyl groups containing 1 to 6 carbon atoms.

As used herein “1,3-diamine” refers to compounds having two nitrogen atoms separated by three optionally substituted atoms, more commonly, three optionally substituted atoms carbon atoms. A 1,3-diamine may form part of a ring structure or may form part of a linear chemical chain.

A “1,3-diamine containing compound” refers to any compound comprising a 1,3-diamine. Included within the definition of “1,3-diamine containing compound” are azepines, 3-oxohexahydro-1H-azepin, and acyclic 1,3-diamines.

As used herein, “acyclic 1,3-diamines” refer to compounds having two nitrogen atoms separated by three optionally substituted atoms, more commonly, three carbon atoms. By way of example, the following fragments constitute acyclic 1,3-diamines:

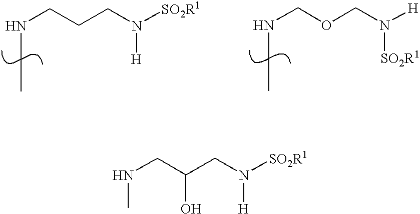

Potential agonists to TRPV4 channel receptors include but are not limited to compounds included in the class of 3-oxohexahydro-1H-azepin, azepine and acyclic 1,3-diamine. Potential agonists also include derivatives of these compounds.

In a one aspect of the invention, TRPV4 channel receptor agonists are selected from those disclosed in International Patent Applications WO 00/38687 (SmithKline Beecham Corporation), WO 01/95911 and WO 02/17924. In addition, agonists may be selected from compounds according to the following formula I, and are referred to herein as azepines: 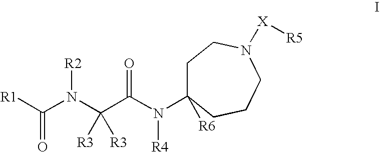
wherein:
R1 is optionally substituted C₃₋₇cycloalkyl, optionally substituted C₃₋₇cycloalkenyl, optionally substituted Het-C₃₋₇alkyl, optionally substituted Het-C₃₋₇alkenyl, optionally substituted aryl, optionally substituted heterocycloalkyl, optionally substituted heteroaryl, or optionally substituted indenyl;
R2 is H, optionally substituted C₁₋₆alkyl, C₃₋₆cycloalkyl-C₀₋₆alkyl, Ar—C₀₋₆alkyl, or Het-C₀₋₆alkyl;
each R3 is independently H, optionally substituted C₁₋₈alkyl, optionally substituted C₂₋₈alkenyl, optionally substituted C₂₋₈alkynyl, Het-C₁₋₆ alkyl, optionally substituted C₃₋₆cycloalkyl, optionally substituted heterocycloalkyl, optionally substituted aryl, or optionally substituted heteroaryl, or optionally substituted C₁-C₆ alkoxy;
R4 is H, or optionally substituted C₁-C₄ alkyl;
R5 is H, optionally substituted C₁₋₈alkyl, optionally substituted C₂₋₈alkenyl, optionally substituted C₂₋₈alkynyl, optionally substituted C₃₋₆cycloalkyl, optionally substituted heterocycloalkyl, optionally substituted aryl, or optionally substituted heteroaryl;
R6 is H or C₁₋₆alkyl; and
X is SO₂, CO, CH₂, or CONH,
and pharmaceutically acceptable salts, hydrates, solvates, and pro-drugs thereof.

International Patent Application WO 00/38687, discloses other potential agonists to a TRPV4 channel receptor which include, but are not limited to, the following 3-oxohexahydro-1H-azepin:
N-{(1S)-1-[({(4R)-1-[(4-chlorophenyl)sulfonyl]-3-oxohexahydro-1H-azepin-4-yl}amino)carbonyl]-3-methylbutyl}-1-benzothiophene-2-carboxamide;
N-{(1S)-1-[({(4R)-1-[(4-fluorophenyl)sulfonyl]-3-oxohexahydro-1H-azepin-4-yl}amino)carbonyl]-3-methylbutyl}-1-benzothiophene-2-carboxamide;
N-{(1S)-1-[({(4R)-1-[(2-cyanophenyl)sulfonyl]-3-oxohexahydro-1H-azepin-4-yl}amino)carbonyl]-3-methylbutyl}-1-methyl-1H-indole-2-carboxamide; and
N-{(1S)-1-[({(4R)-1-[(2-cyanophenyl)sulfonyl]hexahydro-1H-azepin-4-yl}amino)carbonyl]-3-methylbutyl}-1-methyl-1H-indole-2-carboxamide.

The above-referenced 3-oxohexahydro-1H-azepin compounds are those compounds of the Formula (II) or a pharmaceutically acceptable salt or solvate thereof and are referred to herein as 3-oxohexahydro-1H-azepin: 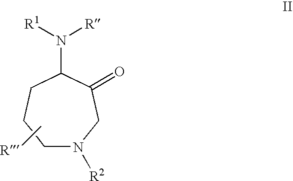
wherein:

R¹ is selected from the group consisting of:

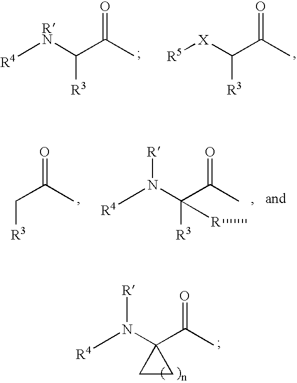

R² is selected from the group consisting of: H, C₁₋₆alkyl, C₃₋₆cycloalkyl-C₀₋₆alkyl, Ar—C₀₋₆alkyl, Het-C₀₋₆alkyl, R⁹C(O)—, R⁹C(S)—, R⁹SO₂—, R⁹OC(O)—, R⁹R¹¹NC(O)—, R⁹R¹¹NC(S)—, R⁹(R¹¹)NSO₂— 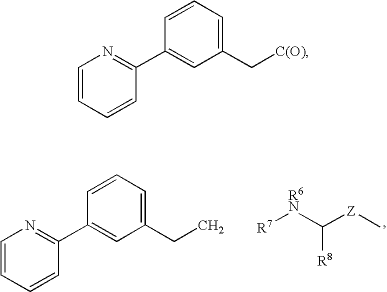
and R⁹SO₂R¹¹NC(O)—;

R³ is selected from the group consisting of: H, C₁₋₆alkyl, C₃₋₆cycloalkyl-C₀₋₆alkyl, C₂₋₆alkenyl, C₂₋₆alkynyl, HetC₀₋₆alkyl and ArC₀₋₆alkyl;

R³ and R′ may be connected to form a pyrrolidine, piperidine or morpholine ring;

R⁴ is selected from the group consisting of: H, C₁₋₆alkyl, C₃₋₆cycloalkyl-C₀₋₆alkyl, Ar—C₀₋₆alkyl, Het-C₀₋₆alkyl, R⁵C(O)—, R⁵C(S)—, R⁵SO₂—, R⁵OC(O)—, R⁵R¹²NC(O)—, and R⁵R¹²NC(S)—;

R⁵ is selected from the group consisting of: H, C₁₋₆alkyl, C₂₋₆alkenyl, C₂₋₆alkynyl, C₃₋₆cycloalkyl-C₀₋₆alkyl, Ar—C₀₋₆alkyl and Het-C₀₋₆alkyl;

R⁶ is selected from the group consisting of: H, C₁₋₆alkyl, Ar—C₀₋₆alkyl, and Het-C₀₋₆alkyl;

R⁷ is selected from the group consisting of: H, C₁₋₆alkyl, C₃₋₆cycloalkyl-C₀₋₆alkyl, Ar—C₀₋₆alkyl, Het-C₀₋₆alkyl, R¹OC(O)—, R¹OC(S)—, R¹⁰SO₂—, R¹⁰OC(O)—, R¹⁰R¹³NC(O)—, and R¹⁰R¹³NC(S)—;

R⁸ is selected from the group consisting of: H, C₁₋₆alkyl, C₂₋₆alkenyl, C₂₋₆alkynyl, HetC₀₋₆alkyl and ArC₀₋₆alkyl;

R⁹ is selected from the group consisting of: C₁₋₆alkyl, C₃₋₆cycloalkyl-C₀₋₆alkyl, Ar—C₀₋₆alkyl and Het-C₀₋₆alkyl;

R¹⁰ is selected from the group consisting of: C₁₋₆alkyl, C₃₋₆cycloalkyl-C₀₋₆alkyl, Ar—C₀₋₆alkyl and Het-C₀₋₆alkyl;

R¹¹ is selected from the group consisting of: H, C₁₋₆alkyl, Ar—C₀₋₆alkyl, and Het-C₀₋₆alkyl;

R¹² is selected from the group consisting of: H, C₁₋₆alkyl, Ar—C₀₋₆alkyl, and Het-C₀₋₆alkyl;

R¹³ is selected from the group consisting of: H, C₁₋₆alkyl, Ar—C₀₋₆alkyl, and Het-C₀₋₆alkyl;

R′ is selected from the group consisting of: H, C₁₋₆alkyl, Ar—C₀₋₆alkyl, and Het-C₀₋₆alkyl;

R″ is selected from the group consisting of: H, C₁₋₆alkyl, Ar—C₀₋₆alkyl, or Het-C₀₋₆alkyl;

R′″ is selected from the group consisting of: H, C₁₋₆alkyl, C₃₋₆cycloalkyl-C₀₋₆alkyl, Ar—C₀₋₆alkyl, and Het-C₀₋₆alkyl;

R″″ is selected from the group consisting of: C₁₋₆alkyl, C₃₋₆cycloalkyl-C₀₋₆alkyl C₂₋₆alkenyl, C₂₋₆alkynyl, HetC₀₋₆alkyl and ArC₀₋₆alkyl;

X is selected from the group consisting of: CH₂, S, and O;

Z is selected from the group consisting of: C(O) and CH₂;

n is an integer from 1 to 5;

and pharmaceutically acceptable salts, hydrates and solvates thereof.

In compounds of Formula II, when R¹ is

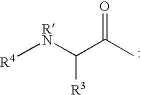

R³ may be selected from the group consisting of: H, C₁₋₆alkyl, C₃₋₆cycloalkyl-C₀₋₆alkyl, C₂₋₆alkenyl, C₂₋₆alkynyl, Het-C₀₋₆alkyl and Ar—C₀₋₆alkyl;

R³ may also be selected from the group consisting of:

H, methyl, ethyl, n-propyl, prop-2-yl, n-butyl, isobutyl, but-2-yl, cyclopropylmethyl, cyclohexylmethyl, 2-methanesulfinyl-ethyl, 1-hydroxyethyl, toluoyl, naphthalen-2-ylmethyl, benzyloxymethyl, and hydroxymethyl.

R³ may also be selected from the group consisting of: toluoyl, isobutyl and cyclohexylmethyl.

R³ may be obutyl.

R⁴ is selected from the group consisting of: H, C₁₋₆alkyl, C₃₋₆cycloalkyl-C₀₋₆alkyl, Ar—C₀₋₆alkyl, Het-C₀₋₆alkyl, R⁵C(O)—, R⁵C(S)—, R⁵SO₂—, R⁵OC(O)—, R⁵R¹³NC(O)—, and R⁵R¹³NC(S)—.

In some embodiments, R⁴ may be methanesulfonyl.

R⁵ may be selected from the group consisting of: H, C₁₋₆alkyl, C₂₋₆alkenyl, C₂₋₆alkynyl, C₃₋₆cycloalkyl-C₀₋₆alkyl, Ar—C₀₋₆alkyl or Het-C₀₋₆alkyl.

When R⁴ is R⁵C(O)—, R⁵ may be selected from the group consisting of: methyl, halogenated methyl, trifluoromethyl, C₁₋₆alkoxy substituted methyl, phenoxy-methyl, 4-fluoro-phenoxy-methyl, heterocycle substituted methyl, 2-thiophenyl-methyl;

ethyl, piperidin-1-yl-ethyl;

butyl, aryl substituted butyl, 4-(4-methoxy)phenyl-butyl;

isopentyl;

cyclohexyl;

pentanonyl, 4-pentanonyl;

butenyl, aryl substituted butenyl, 4,4-bis(4-methoxyphenyl)-but-3-enyl;

acetyl;

phenyl, phenyl substituted with one or more halogens, 3,4-dichlorophenyl and 4-fluorophenyl, phenyl substituted with one or more aryloxy or C₁₋₆alkoxy groups, 3,4-dimethoxy-phenyl, 3-benzyloxy-4-methoxy-phenyl, phenyl substituted with one or more C₁₋₆alkyl sulfonyl groups, 4-methanesulfonyl-phenyl;

benzyl;

naphthalenyl, naphthylen-2-yl;

benzo[1,3]dioxolyl, benzo[1,3]dioxol-5-yl;

furanyl, furan-2-yl, substituted furanyl, such as 5-nitro-furan-2-yl, 5-(4-nitrophenyl)-furan-2-yl, 5-(3-trifluoromethyl-phenyl)-furan-2-yl, halogen substituted furanyl, 5-bromo-furan-2-yl, aryl substituted furanyl, 5-(4-chloro-phenyl)-furan-2-yl, C₁₋₆alkyl substituted furanyl, 3-methyl-furan-2-yl, 4-methyl-furan-2-yl, 2,5-dimethyl-furan-2-yl, and 2,4-dimethyl-furan-3-yl;

tetrahydrofuranyl, tetrahydrofuran-2-yl;

benzofuranyl, benzofuran-2-yl, and substituted benzofuranyl, 5-(2-piperazin-4-carboxylic acid tert-butyl ester-ethoxy)benzofuran-2-yl, 5-(2-morpholino-4-yl-ethoxy)-benzofuran-2-yl, 5-(2-piperazin-1-yl-ethoxy)benzofuran-2-yl, 5-(2-cyclohexyl-ethoxy)-benzofuran-2-yl; C₁₋₆alkoxy substituted benzofuranyl, 7-methoxy-benzofuran-2-yl, 5-methoxy-benzofuran-2-yl, 5,6-dimethoxy-benzofuran-2-yl, halogen substituted benzofuranyl, 5-fluoro-benzofuran-2-yl, 5,6-difluoro-benzofuran-2-yl, C₁₋₆alkyl substituted benzofuranyl, 3-methyl-benzofuran-2-yl, 3,5-dimethyl-benzofuran-2-yl, and 3-ethyl-benzofuran-2-yl; also 5-fluoro-3-methyl-benzofuran-2-yl, 6-fluoro-3-methyl-benzofuran-2-yl, 5-methoxy-3-methyl-benzofuran-2-yl, 4-methoxy-3-methyl-benzofuran-2-yl, and 6-methoxy-3-methyl-benzofuran-2-yl;

naphtho[2,1-b]-furanyl, naphtho[2,1-b]-furan-2-yl, alkyl substituted naphtho[2,1-b]-furanyl, 1-methyl-naphtho[2,1-b]-furan-2-yl;

benzo[b]thiophenyl, benzo[b]thiophen-2-yl; C₁₋₆alkoxy substituted benzo[b]thiophenyl, 5,6-dimethoxy-benzo[b]thiophen-2-yl;

quinolinyl, quinolin-2-yl, quinolin-3-yl, quinolin-4-yl, quinolin-6-yl, and quinolin-8-yl;

quinoxalinyl, quinoxalin-2-yl;

1,8 naphthyridinyl, 1,8 naphthyridin-2-yl;

indolyl, indol-2-yl, indol-6-yl, indol-5-yl, C₁₋₆alkyl substituted indolyl, N-methyl-indol-2-yl;

pyridinyl, pyridin-2-yl, pyridin-3-yl, pyridin-5-yl, C₁₋₆alkyl substituted pyridinyl, 2-methyl-pyridin-5-yl, and oxy-pyridinyl, 1-oxy-pyridin-2-yl and 1-oxy-pyridin-3-yl;

furo[3,2-b]-pyridinyl, furo[3,2-b]-pyridin-2-yl, C₁₋₆alkyl substituted furo[3,2-b]-pyridinyl, 3-methyl-furo[3,2-b]-pyridin-2-yl;

thiophenyl, thiophen-3-yl, also thiophen-2-yl, C₁₋₆alkyl substituted thiophenyl, 5-methyl-thiophen-2-yl and 5-methyl-thiophen-3-yl, halogen substituted thiophenyl, 4,5-dibromo-thiophen-2-yl;

thieno[3,2-b]thiophene, thieno[3,2-b]thiophene-2-yl, C₁₋₆alkyl substituted thieno[3,2-b]thiophene-2-yl, 5-tert-butyl-3-methyl-thieno[3,2-b]thiophene-2-yl;

isoxazolyl, isoxazol-4-yl, C₁₋₆alkyl substituted isoxazolyl, 3,5-dimethyl-isoxazol-4-yl;

oxazolyl, oxazol-4-yl, 5-methyl-2-phenyl oxazol-4-yl, 2-phenyl-5-trifluoromethyl-oxazol-4-yl; and

1H-benzoimidazolyl, 1H-benzoimidazol-5-yl.

When R⁴ is R⁵SO₂, R⁵ may be pyridin-2-yl or 1-oxo-pyridin-2-yl.

R′ is selected from the group consisting of: H, C₁₋₆alkyl, Ar—C₀₋₆alkyl, and Het-C₀₋₆alkyl.

R′ may also be selected from the group consisting of: H and naphthalen-2-yl-methyl.

R″ is selected from the group consisting of: H, C₁₋₆alkyl, Ar—C₀₋₆alkyl, and Het-C₀₋₆alkyl.

R′″ is selected from the group consisting of: H, C₁₋₆alkyl, C₃₋₆cycloalkyl-C₀₋₆alkyl, and Het-C₀₋₆alkyl.

R″″ may also be selected from the group consisting of: H, methyl and 6,6-dimethyl.

In compounds of Formula II, when R¹ is 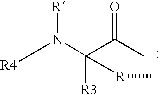
R³ is selected from the group consisting of: C₁₋₆alkyl, C₃₋₆cycloalkyl-C₀₋₆alkyl, C₂₋₆alkenyl, C₂₋₆alkynyl, Het-C₀₋₆alkyl and Ar—C₀₋₆alkyl.

R³ may also be selected from the group consisting of methyl, ethyl, n-propyl, n-butyl, isobutyl, t-butyl, cyclohexylmethyl, and toluoyl.

R′″ is selected from the group consisting of: C₁₋₆alkyl, C₃₋₆cycloalkyl-C₀₋₆alkyl, C₂₋₆alkenyl, C₂₋₆alkynyl, HetC₀₋₆alkyl and ArC₀₋₆alkyl;

R″″ may also be selected from the group consisting of methyl, ethyl, n-propyl, n-butyl, isobutyl and t-butyl.

R″″ may be methyl.

In such compounds, R, R″, R′″, R⁴, and R⁵ are as described above wherein

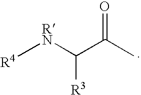

In compounds of Formula II, when R¹ is

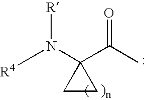

n may be an integer of from 1 to 5; and

R′, R″, R′″, R⁴, and R⁵ are as described above wherein

R¹ is

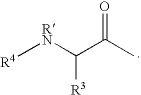

n may be 3.

The ring may be unsubstituted or substituted with one or more of C₁₋₆alkyl, C₃₋₆cycloalkyl-C₀₋₆alkyl, C₂₋₆alkenyl, C₂₋₆alkynyl, HetC₀₋₆alkyl, ArC₀₋₆alkyl, or halogen.

The ring may be unsubstituted.

In compounds of Formula II, R² is selected from the group consisting of:
H, C₁₋₆alkyl, C₃₋₆cycloalkyl-C₀₋₆alkyl, Ar—C₀₋₆alkyl, Het-C₀₋₆alkyl, R⁹C(O)—, R⁹C(S)—, R⁹SO₂—, R⁹OC(O)—, R⁹R¹¹NC(O)—, R⁹R¹¹NC(S)—, R⁹R¹¹NSO₂—, 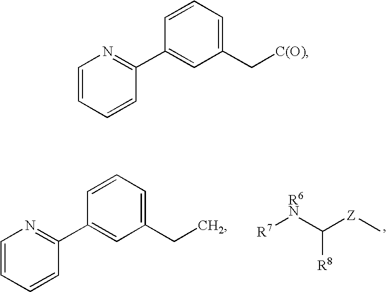
and R⁹SO₂R¹¹NC(O)—.

In such embodiments:

R⁶ may be selected from the group consisting of: H, C₁₋₆alkyl, Ar—C₀₋₆alkyl, or Het-C₀₋₆alkyl, H.

R⁷ is selected from the group consisting of: H, C₁₋₆alkyl, C₃₋₆cycloalkyl-C₀₋₆alkyl, Ar—C₀₋₆alkyl, Het-C₀₋₆alkyl, R¹⁰C(O)—, R¹OC(S)—, R¹⁰SO₂—, R¹⁰OC(O)—, R¹⁰R¹⁴NC(O)—, R¹⁰R¹⁴NC(S)—, R⁷ or R¹⁰OC(O).

R⁸ is selected from the group consisting of: H, C₁₋₆alkyl, C₂₋₆alkenyl, C₂₋₆alkynyl, HetC₀₋₆alkyl and ArC₀₋₆alkyl; C₁₋₆alkyl, or isobutyl.

R⁹ is selected from the group consisting of: C₁₋₆alkyl, C₃₋₆cycloalkyl-C₀₋₆alkyl, Ar—C₀₋₆alkyl, and Het-C₀₋₆alkyl.

R⁹ may also be selected from the group consisting of:

methyl; ethyl, C₁₋₆alkyl-substituted ethyl, 2-cyclohexyl-ethyl;

propyl; butyl, C₁₋₆butyl, 3-methylbutyl; tert-butyl,

when R² is R⁹OC(O); isopentyl;

phenyl, halogen substituted phenyl, 3,4-dichlorophenyl, 4-bromophenyl, 2-fluorophenyl, 3-fluorophenyl, 4-fluorophenyl, 2-chlorophenyl, 3-chlorophenyl, 4-chlorophenyl, C₁₋₆alkoxy phenyl 3-methoxyphenyl, 4-methoxyphenyl, 3,4-dimethoxyphenyl, cyanophenyl, 2-cyanophenyl; C₁₋₆alkyl substituted phenyl, 4-ethyl-phenyl, 2-methyl phenyl, 4-methyl phenyl, C₁₋₆alkyl sulfonyl substituted phenyl, 4-methanesulfonyl phenyl, and 2-methanesulfonyl phenyl;

toluoyl, Het-substituted toluoyl, 3-(pyridin-2-yl)toluoyl;

naphthylene, naphthyl-2-ene;

benzoic acid, 2-benzoic acid;

benzo[1,3]dioxolyl, benzo[1,3]dioxol-5-yl;

benzo[1,2,5]oxadiazolyl, benzo[1,2,5]oxadiazol-4-yl;

pyridinyl, pyridin-2-yl, pyridin-3-yl, 1-oxy-pyridinyl, 1-oxy-pyridin-2-yl, 1-oxy-pyridin-3-yl; C₁₋₆alkylpyridinyl, 3-methyl-pyridin-2-yl, 6-methyl-pyridin-2-yl;

thiophenyl, thiophenyl-2-yl;

thiazolyl, thiazol-2-yl;

1H-imidazolyl, 1H-imidazol-2-yl, 1H-imidazol-4-yl, C₁₋₆alkyl substituted imidazolyl, 1-methyl-1H-imidazol-2-yl, 1-methyl-1H-imidazol-4-yl, and 1,2-dimethyl-1H-imidazol-4-yl;

triazolyl, 1H-[1,2,4]triazolyl, 1H-[1,2,4]triazol-3-yl, C₁₋₆alkyl substituted 1H-[1,2,4]triazolyl, 5-methyl-1H-[1,2,4]triazol-3-yl; and

isoxazolyl, isoxazol-4-yl, C₁₋₆alkyl substituted isoxazolyl, 3,5-dimethyl-isoxazol-4-yl.

When R² is R⁹SO₂, R⁹ may be selected from the group consisting of: pyridin-2-yl and 1-oxy-pyridin-2-yl.

When R² is R⁹SO₂R¹¹NC(O)—, R⁹ may be Ar—C₀₋₆alkyl, Ar, substituted phenyl such as 2-methyl phenyl, 4-methyl phenyl, 2-chloro phenyl, and 4-fluoro phenyl.

When R² is R⁹C(O)—, R⁹ may be selected from the group consisting of C₁₋₆alkyl, C₃₋₆cycloalkyl-C₀₋₆alkyl, and Het-C₀₋₆alkyl, 1-oxy-pyridin-2-yl, cyclohexyl ethyl, and 3-methyl butyl.

R¹¹ is selected from the group consisting of: H, C₁₋₆alkyl, Ar—C₀₋₆alkyl, and Het-C₀₋₆alkyl.

When R² is R⁹SO₂R¹¹NC(O)—, R¹¹ may be H.

When R² is Ar—C₀₋₆alkyl, R² may be phenyl, substituted phenyl, halogen substituted phenyl, 2-fluorobenzyl.

When R² is C₁₋₆alkyl, R² may be selected from 1-propyl, 1-butyl, and 1-pentyl.

When R² is Het-C₀₋₆alkyl, Het-C₀₋₆alkyl may be Het-methyl, and Het in Het-methyl may be selected from the group consisting of:

pyridinyl, pyridin-2-yl, C₁₋₆alkylpyridinyl, 6-methyl-pyridin-2-yl;

thiophenyl, thiophene-2-yl, thiophen-2-yl or benzo[b]thiophen-2-yl;

thiazolyl, thiazol-4-yl such as 1-(2-morpholin-4-yl-thiazol-4-yl), and 1-(isothiazol-3-yl);

1H-imidazolyl, 1H-imidazol-2-yl, 1H-imidazol-4-yl, C₁₋₆alkyl substituted imidazolyl, 1-methyl-1H-imidazol-2-yl;

triazolyl, 3H-[1,2,3]triazolyl, 3H-[1,2,3]triazol-4-yl, C₁₋₆alkyl substituted 3H-[1,2,3]triazolyl, 3-phenyl-3H-[1,2,3]triazolyl-4-yl;

quinolinyl, quinolin-2-yl, quinolin-2-yl;

furanyl, furan-2-yl, substituted furanyl, such as 5-ethyl-furan-2-yl;

thieno[3,2-b]thiophene, thieno[3,2-b]thiophene-2-yl, C₁₋₆alkyl substituted thieno[3,2-b]thiophenyl, 3,4-dimethyl-thieno[3,2-b]thiophene-2-yl.

R² may be:

H;

toluoyl;

aryl substituted ethyl, 2-phenyl ethyl, 2-[3-(pyridin-2-yl)phenyl]ethyl.

In compounds of Formula II wherein:

R¹ is

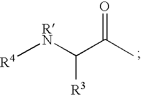

R² may be selected from the group consisting of: Ar—C₀₋₆alkyl, R⁹C(O)—, R⁹SO₂, R⁹R¹¹NC(O)—, and

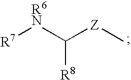

R³ is selected from the group consisting of: H, C₁₋₆alkyl, C₃₋₆cycloalkyl-C₀₋₆alkyl and Ar—C₀₋₆alkyl;

R⁴ is selected from the group consisting of: R⁵OC(O)—, R⁵C(O)— and R⁵SO₂—;

R⁵ is selected from the group consisting of: C₁₋₆alkyl, Ar—C₀₋₆alkyl and Het-C₀₋₆alkyl;

R⁶ is H;

R⁷ is R¹⁰OC(O);

R⁸ is C₁₋₆alkyl;

R⁹ is selected from the group consisting of: C₁₋₆alkyl, Ar—C₀₋₆alkyl and Het-C₀₋₆alkyl;

R¹⁰ is selected from the group consisting of: C₁₋₆alkyl, Ar—C₀₋₆alkyl and Het-C₀₋₆alkyl;

R′ is H;

R″ is H;

R′″ is H; and

Z is selected from the group consisting of: C(O) and CH₂.

Compounds of Formula II also include compounds wherein R² is selected from the group consisting of: Ar—C₀₋₆alkyl, R⁹C(O)—, R⁹SO₂.

Compounds of Formula II also include compounds wherein:

R¹ is

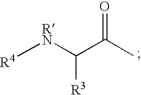

R² is selected from the group consisting of: Ar—C₀₋₆alkyl, R⁹C(O)— and R⁹SO₂;

R³ is selected from the group consisting of: H, methyl, ethyl, n-propyl, prop-2-yl, n-butyl, isobutyl, but-2-yl, cyclopropylmethyl, cyclohexylmethyl, 2-methanesulfinyl-ethyl, 1-hydroxyethyl, toluoyl, naphthalen-2-ylmethyl, benzyloxymethyl, and hydroxymethyl;

R⁴ is R⁵C(O)—;

R⁵ is selected from the group consisting of:

methyl, halogenated methyl, trifluoromethyl, C₁₋₆alkoxy substituted methyl, phenoxy-methyl, 4-fluoro-phenoxy-methyl, heterocycle substituted methyl, 2-thiophenyl-methyl;

ethyl, piperidin-1-yl-ethyl;

butyl, aryl substituted butyl, 4-(4-methoxy)phenyl-butyl;

isopentyl;

cyclohexyl;

pentanonyl, 4-pentanonyl;

butenyl, aryl substituted butenyl, 4,4-bis(4-methoxyphenyl)-but-3-enyl;

acetyl;

phenyl, phenyl substituted with one or more halogens, 3,4-dichlorophenyl and 4-fluorophenyl, phenyl substituted with one or more aryloxy or C₁₋₆alkoxy groups, 3,4-dimethoxy-phenyl, 3-benzyloxy-4-methoxy-phenyl, phenyl substituted with one or more C₁₋₆alkyl sulfonyl groups, 4-methanesulfonyl-phenyl;

benzyl;

naphthalenyl, naphthylen-2-yl;

benzo[1,3]dioxolyl, benzo[1,3]dioxol-5-yl;

furanyl, furan-2-yl, substituted furanyl, such as 5-nitro-furan-2-yl, 5-(4-nitrophenyl)-furan-2-yl, 5-(3-trifluoromethyl-phenyl)-furan-2-yl, halogen substituted furanyl, even 5-bromo-furan-2-yl, aryl substituted furanyl, even 5-(4-chloro-phenyl)-furan-2-yl, C₁₋₆alkyl substituted furanyl, even 3-methyl-furan-2-yl, 4-methyl-furan-2-yl, 2,5-dimethyl-furan-2-yl, and 2,4-dimethyl-furan-3-yl;

tetrahydrofuranyl, tetrahydrofuran-2-yl;

benzofuranyl, benzofuran-2-yl, and substituted benzofuranyl, 5-(2-piperazin-4-carboxylic acid tert-butyl ester-ethoxy)benzofuran-2-yl, 5-(2-morpholino-4-yl-ethoxy)-benzofuran-2-yl, 5-(2-piperazin-1-yl-ethoxy)benzofuran-2-yl, 5-(2-cyclohexyl-ethoxy)-benzofuran-2-yl; C₁₋₆alkoxy substituted benzofuranyl, 7-methoxy-benzofuran-2-yl, 5-methoxy-benzofuran-2-yl, 5,6-dimethoxy-benzofuran-2-yl, halogen substituted benzofuranyl, 5-fluoro-benzofuran-2-yl, 5,6-difluoro-benzofuran-2-yl, C₁₋₆alkyl substituted benzofuranyl, 3-methyl-benzofuran-2-yl, 3,5-dimethyl-benzofuran-2-yl, and 3-ethyl-benzofuran-2-yl; also 5-fluoro-3-methyl-benzofuran-2-yl, 6-fluoro-3-methyl-benzofuran-2-yl, 5-methoxy-3-methyl-benzofuran-2-yl, 4-methoxy-3-methyl-benzofuran-2-yl, and 6-methoxy-3-methyl-benzofuran-2-yl;

naphtho[2,1-b]-furanyl, naphtho[2,1-b]-furan-2-yl, alkyl substituted naphtho[2,1-b]-furanyl, 1-methyl-naphtho[2,1-b]-furan-2-yl;

benzo[b]thiophenyl, benzo[b]thiophen-2-yl; C₁₋₆alkoxy substituted benzo[b]thiophenyl, 5,6-dimethoxy-benzo[b]thiophen-2-yl;

quinolinyl, quinolin-2-yl, quinolin-3-yl, quinolin-4-yl, quinolin-6-yl, and quinolin-8-yl;

quinoxalinyl, quinoxalin-2-yl;

1,8 naphthyridinyl, 1,8 naphthyridin-2-yl;

indolyl, indol-2-yl, indol-6-yl, indol-5-yl, C₁₋₆alkyl substituted indolyl, N-methyl-indol-2-yl;

pyridinyl, pyridin-2-yl, pyridin-3-yl, pyridin-5-yl, C₁₋₆alkyl substituted pyridinyl, 2-methyl-pyridin-5-yl, and oxy-pyridinyl, 1-oxy-pyridin-2-yl and 1-oxy-pyridin-3-yl;

furo[3,2-b]-pyridinyl, furo[3,2-b]-pyridin-2-yl, C₁₋₆alkyl substituted furo[3,2-b]-pyridinyl, 3-methyl-furo[3,2-b]-pyridin-2-yl;

thiophenyl, thiophen-3-yl, also thiophen-2-yl, C₁₋₆alkyl substituted thiophenyl, 5-methyl-thiophen-2-yl and 5-methyl-thiophen-3-yl, halogen substituted thiophenyl, 4,5-dibromo-thiophen-2-yl;

thieno[3,2-b]thiophene, thieno[3,2-b]thiophene-2-yl, C₁₋₆alkyl substituted thieno[3,2-b]thiophene-2-yl, 5-tert-butyl-3-methyl-thieno[3,2-b]thiophene-2-yl;

isoxazolyl, isoxazol-4-yl, C₁₋₆alkyl substituted isoxazolyl, 3,5-dimethyl-isoxazol-4-yl;

oxazolyl, oxazol-4-yl, 5-methyl-2-phenyl oxazol-4-yl, 2-phenyl-5-trifluoromethyl-oxazol-4-yl; and

1H-benzoimidazolyl, 1H-benzoimidazol-5-yl.

R⁹ is selected from the group consisting of:

methyl;

ethyl, C₁₋₆alkyl-substituted ethyl, 2-cyclohexyl-ethyl;

propyl;

butyl, C₁₋₆butyl, 3-methylbutyl;

tert-butyl, particularly when R² is R⁹OC(O);

isopentyl;

phenyl, halogen substituted phenyl, 3,4-dichlorophenyl, 4-bromophenyl, 2-fluorophenyl, 3-fluorophenyl, 4-fluorophenyl, 2-chlorophenyl, 3-chlorophenyl, 4-chlorophenyl, C₁₋₆alkoxy phenyl, 3-methoxyphenyl, 4-methoxyphenyl, 3,4-dimethoxyphenyl, cyanophenyl, 2-cyanophenyl; C₁₋₆alkyl substituted phenyl, 4-ethyl-phenyl, 2-methyl phenyl, 4-methyl phenyl, C₁₋₆alkyl sulfonyl substituted phenyl, 4-methanesulfonyl phenyl, and 2-methanesulfonyl phenyl;

toluoyl, Het-substituted toluoyl, 3-(pyridin-2-yl)toluoyl;

naphthylene, naphthyl-2-ene;

benzoic acid, 2-benzoic acid;

benzo[1,3]dioxolyl, benzo[1,3]dioxol-5-yl;

benzo[1,2,5]oxadiazolyl, benzo[1,2,5]oxadiazol-4-yl;

pyridinyl, pyridin-2-yl, pyridin-3-yl, 1-oxy-pyridinyl, 1-oxy-pyridin-2-yl, 1-oxy-pyridin-3-yl; C₁₋₆alkylpyridinyl, 3-methyl-pyridin-2-yl, 6-methyl-pyridin-2-yl;

thiophenyl, thiophenyl-2-yl;

thiazolyl, thiazol-2-yl;

1H-imidazolyl, 1H-imidazol-2-yl, 1H-imidazol-4-yl, C₁₋₆alkyl substituted imidazolyl, even 1-methyl-1H-imidazol-2-yl, 1-methyl-1H-imidazol-4-yl, and 1,2-dimethyl-1H-inmidazol-4-yl;

triazolyl, 1H-[1,2,4]triazolyl, 1H-[1,2,4]triazol-3-yl, C₁₋₆alkyl substituted 1H-[1,2,4]triazolyl, 5-methyl-1H-[1,2,4]triazol-3-yl; and

isoxazolyl, isoxazol-4-yl, C₁₋₆alkyl substituted isoxazolyl, 3,5-dimethyl-isoxazol-4-yl.

R′ is H;

R″ is H; and

R′″ is H.

Compounds of Formula I also include compounds wherein:

R¹ is

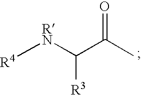

R² is R⁹SO₂;

R³ is isobutyl;

R⁴ is R⁵C(O);

R⁵ is selected from the group consisting of: 3-methyl-benzofuran-2-yl, thieno[3,2-b]thiophen-2-yl, 5-methoxybenzofuran-2-yl, quinoxalin-2-yl, and quinolin-2-yl, 3-methyl-benzofuran-2-yl;

R⁹ is selected from the group consisting of: pyridin-2-yl and 1-oxy-pyridin-2-yl, 1-oxy-pyridin-2-yl.

R′ is H; and

R″ is H;

R⁵ may be 3-methyl-benzofuran-2-yl; and

R⁹ may be 1-oxy-pyridin-2-yl.

Compounds of Formula II can also be provided as compounds of Formula Ia: 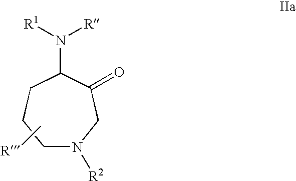
wherein:

R¹ is selected from the group consisting of:

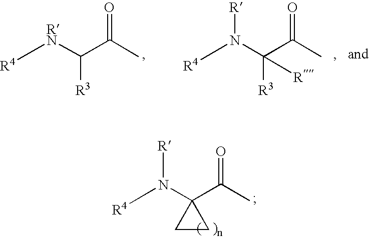

R² is selected from the group consisting of: C₁₋₆alkyl, Ar—C₀₋₆alkyl, Het-C₀₋₆alkyl, R⁹C(O)—, R⁹SO₂—, R⁹R¹¹NC(O)—, and R⁹SO₂R¹¹NC(O)—;

R³ is selected from the group consisting of: C₁₋₆alkyl, C₃₋₆cycloalkyl-C₀₋₆alkyl, C₂₋₆alkenyl, C₂₋₆alkynyl, Het-C₀₋₆alkyl and Ar—C₀₋₆alkyl, C₁₋₆alkyl;

R³ and R′ may be connected to form a pyrrolidine, piperidine or morpholine ring;

R⁴ is R⁵C(O)—;

R⁵ is selected from the group consisting of: C₁₋₆alkyl and Het-C₀₋₆alkyl, Het-C₀₋₆alkyl;

R⁹ is selected from the group consisting of: C₁₋₆alkyl, C₃₋₆cycloalkyl-C₀₋₆alkyl, Ar—C₀₋₆alkyl and Het-C₀₋₆alkyl;

R¹¹ is selected from the group consisting of: H, C₁₋₆alkyl, Ar—C₀₋₆alkyl and Het-C₀₋₆alkyl, H;

R′ is H;

R″ is H;

R′″ is selected from the group consisting of: H and C₁₋₆alkyl, H;

R″″ is selected from the group consisting of: C₁₋₆alkyl, C₃₋₆cycloalkyl-C₀₋₆alkyl C₂₋₆alkenyl, C₂₋₆alkynyl, HetC₀₋₆alkyl and ArC₀₋₆alkyl; and

n is an integer from 1 to 5;

and pharmaceutically acceptable salts, hydrates and solvates thereof.

In Formula Ia, when R¹ is

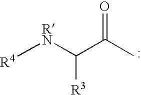

R³ may be C₁₋₆alkyl;

R³ may be selected from the group consisting of: but-2-yl and isobutyl.

R⁴ is R⁵C(O)—.

R⁵ may be selected from the group consisting of: C₁₋₆alkyl and Het-C₀₋₆alkyl, Het-C₁₋₆alkyl;

R⁵ may also be from the group consisting of:

piperidin-ethyl, piperidin-1-yl-ethyl;

benzo[1,3]dioxolyl, benzo[1,3]dioxol-5-yl;

furanyl, furan-2-yl, aryl substituted furanyl, such as 5-(3-trifluoromethyl-phenyl)-furan-2-yl, C₁₋₆alkyl substituted furanyl, even 3-methyl-furan-2-yl, 4-methyl-furan-2-yl, 2,5-dimethyl-furan-2-yl, and 2,4-dimethyl-furan-3-yl;

benzofuranyl, benzofuran-2-yl, C₁₋₆alkoxy substituted benzofuranyl, 5-methoxy-benzofuran-2-yl, halogen substituted benzofuranyl, 5-fluoro-benzofuran-2-yl, C₁₋₆alkyl substituted benzofuranyl, 3-methyl-benzofuran-2-yl, 3,5-dimethyl-benzofuran-2-yl, and 3-ethyl-benzofuran-2-yl; also 5-fluoro-3-methyl-benzofuran-2-yl, 5-methoxy-3-methyl-benzofuran-2-yl, 4-methoxy-3-methyl-benzofuran-2-yl, and 6-methoxy-3-methyl-benzofuran-2-yl;

naphtho[2,1-b]-furanyl, naphtho[2,1-b]-furan-2-yl, C₁₋₆alkyl substituted naphtho[2,1-b]-furanyl, 1-methyl-naphtho[2,1-b]-furan-2-yl;

benzo[b]thiophenyl, benzo[b]thiophen-2-yl;

quinolinyl, quinolin-2-yl;

quinoxalinyl, quinoxalin-2-yl;

pyridinyl, pyridin-2-yl, pyridin-3-yl, pyridin-5-yl, and oxy-pyridinyl, 1-oxy-pyridin-2-yl and 1-oxy-pyridin-3-yl;

furo[3,2-b]-pyridinyl, furo[3,2-b]-pyridin-2-yl, C₁₋₆alkyl substituted furo[3,2-b]-pyridin-2-yl, 3-methyl-furo[3,2-b]-pyridin-2-yl;

thiophenyl, thiophen-3-yl, and thiophen-2-yl, C₁₋₆alkyl substituted thiophenyl, 5-methyl-thiophen-2-yl and 5-methyl-thiophen-3-yl; and

thieno[3,2-b]thiophene, thieno[3,2-b]thiophene-2-yl; and

1H-benzoimidazolyl, 1H-benzoimidazol-5-yl.

R⁵ may be selected from the group consisting of:

3-methyl-benzofuran-2-yl, thieno[3,2-b]thiophen-2-yl, 5-methoxybenzofuran-2-yl, quinoxalin-2-yl, and quinolin-2-yl.

In Formula Ia, when R¹ is

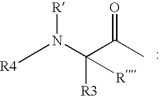

R³ may be selected from the group consisting of: C₁₋₆alkyl, C₃₋₆cycloalkyl-C₀₋₆alkyl, C₂₋₆alkenyl, C₂₋₆alkynyl, Het-C₀₋₆alkyl and Ar—C₀₋₆alkyl.

R³ may also be selected from the group consisting of methyl, ethyl, n-propyl, n-butyl, isobutyl, t-butyl, cyclohexylmethyl, and toluoyl.

R″″ may be selected from the group consisting of: C₁₋₆alkyl, C₃₋₆cycloalkyl-C₀₋₆alkyl, C₂₋₆alkenyl, C₂₋₆alkynyl, HetC₀₋₆alkyl and ArC₀₋₆alkyl;

R″″ may also be selected from the group consisting of methyl, ethyl, n-propyl, n-butyl, isobutyl and t-butyl.

R″″ may be methyl.

In such compounds, R¹ and R⁴ are as described above wherein

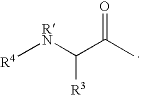

In Formula Ia, when R¹ is

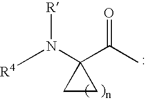

n may be an integer of from 1 to 5; and

R¹ and R⁴ are as described above wherein

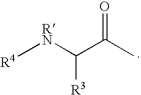

The cyclic ring may be unsubstituted or substituted with one or more of C₁₋₆alkyl, C₃₋₆cycloalkyl-C₀₋₆alkyl, C₂₋₆alkenyl, C₂₋₆alkynyl, HetC₀₋₆alkyl, ArC₀₋₆alkyl, or halogen.

In Formula Ia, R² may be selected from the group consisting of: C₁₋₆alkyl, Ar—C₀₋₆alkyl, Het-C₀₋₆alkyl, R⁹C(O)—, R⁹SO₂—, R⁹R¹¹NC(O)—, and R⁹SO₂R¹¹NC(O)—.

In such embodiment:

R⁹ may be selected from the group consisting of: C₁₋₆alkyl, C₃₋₆cycloalkyl-C₀₋₆alkyl, Ar—C₀₋₆alkyl, and Het-C₀₋₆alkyl.

R⁹ may also be selected from the group consisting of: ethyl, C₁₋₆alkyl-substituted ethyl, 2-cyclohexyl-ethyl;

propyl, prop-1-yl;

isopentyl;

butyl, but-1-yl;

phenyl, halogen substituted phenyl, 3-fluorophenyl, 4-fluorophenyl, 2-chlorophenyl; C₁₋₆alkyl substituted phenyl, 4-ethyl-phenyl, 2-methyl phenyl, 4-methyl phenyl, C₁₋₆alkyl sulfonyl substituted phenyl, 4-methanesulfonyl phenyl, and 2-methanesulfonyl phenyl;

pyridinyl, pyridin-2-yl, 1-oxy-pyridinyl, 1-oxy-pyridin-2-yl;

1H-imidazolyl, 1H-imidazol-2-yl C₁₋₆alkyl substituted imidazolyl, 1-methyl-1H-imidazol-2-yl; and

isoxazolyl, isoxazol-4-yl, C₁₋₆alkyl substituted isoxazolyl, 3,5-dimethyl-isoxazol-4-yl.

When R² is R⁹SO₂, R⁹ may be selected from the group consisting of: pyridin-2-yl and 1-oxy-pyridin-2-yl.

When R² is R⁹SO₂R¹¹NC(O)—, R⁹ may be Ar—C₀₋₆alkyl, Ar, substituted phenyl such as 2-methyl phenyl, 4-methyl phenyl, 2-chloro phenyl, 4-fluoro phenyl.

When R² is R⁹C(O)—, R⁹ may be selected from the group consisting of C₁₋₆alkyl, C₃₋₆cycloalkyl-C₀₋₆alkyl, and Het-C₀₋₆alkyl, 1-oxy-pyridin-2-yl, 2-cyclohexyl ethyl, and isopentyl.

When R² is R⁹SO₂R¹¹NC(O)—, R¹¹ is selected from the group consisting of:

H, C₁₋₆alkyl, Ar—C₀₋₆alkyl and Het-C₀₋₆alkyl. In such embodiment, R¹¹ may be H.

R² may suitably be selected from the group consisting of:

C₁₋₆alkyl, Ar—C₀₋₆alkyl and Het-C₀₋₆alkyl, C₁₋₆alkyl and Het-C₀₋₆alkyl.

When R² is Ar—C₀₋₆alkyl, R² may be phenyl, substituted phenyl, halogen substituted phenyl, 2-fluorobenzyl.

When R² is C₁₋₆alkyl, R² may be selected from 1-propyl, 1-butyl, and 1-pentyl.

When R² is Het-C₀₋₆alkyl, Het-C₀₋₆alkyl may be Het-methyl, and Het in Het-methyl may be selected from the group consisting of:

pyridinyl, pyridin-2-yl, C₁₋₆alkylpyridinyl, 6-methyl-pyridin-2-yl;

thiophenyl, thiophene-2-yl;

benzo[b]thiophen-2-yl;

thiazolyl, thiazol-4-yl such as isothiazol-3-yl;

1H-imidazolyl, 1H-imidazol-2-yl, C₁₋₆alkyl substituted imidazolyl, 1-methyl-1H-imidazol-2-yl;

triazolyl, 3H-[1,2,3]triazolyl, 3H-[1,2,3]triazol-4-yl, C₁₋₆alkyl substituted 3H-[1,2,3]triazolyl, 3-phenyl-3H-[1,2,3]triazolyl-4-yl;

quinolinyl, quinolin-2-yl, quinolin-2-yl;

furanyl, furan-2-yl, substituted furanyl, such as 5-ethyl-furan-2-yl;

thieno[3,2-b]thiophene, thieno[3,2-b]thiophene-2-yl, C₁₋₆alkyl substituted thieno[3,2-b]thiophene-2-yl, 3,4-dimethyl-thieno[3,2-b]thiophene-2-yl.

Other compounds of the instant invention include compounds of the following Formula III, also referred to herein as acyclic 1,3-diamines: 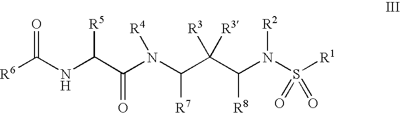
or a pharmaceutically acceptable salt thereof, or a solvate thereof, or a combination thereof, wherein:
R¹ is phenyl, thienyl, furanyl, benzoxadiazolyl, imidazo[2,1-b][1,3]thiazolyl, C₃-C₇ cycloalkyl-C₁-C₄ alkylenyl, C₃-C₇ cycloalkyloxy-C₁-C₄ alkylenyl, or N-ethenyl-tetrahydroindolyl, wherein R¹ is optionally substituted with one or more substituents selected from the group consisting of C₁-C₆ alkyl, C₁-C₆ alkylsulfonyl, [(methylamino)carbonyl]amino, cyano, nitro, trifluoromethyl, trifluoromethoxy, carboC₁-C₆alkyloxy, and halo;
R² is H, C₁-C₆ alkyl, halo C₁-C₆ alkyl, di C₁-C₆ alkylamino-C₁-C₆ alkylenyl, C₁-C₆ alkyloxy-C₁-C₆ alkylenyl, C₁-C₆ alkyloxy, pyridinyl-C₁-C₆ alkylenyl, C₃-C₇ cycloalkyl, or tetrahydropyranyl;
R³ is H, hydroxy, —O—C₁-C₆ alkyl, —SH, —S—C₁-C₆ alkyl, amino, C₁-C₄ alkylamino, propenyloxy, or halo;
R^{3′} is H or C₁-C₆ alkyl, or R^{3′} together with R³ forms an oxo group;
R⁴ is H or C₁-C₆ alkyl;
R⁵ is iso-butyl, 3,3-dimethylbutyl, thiazolylmethylenyl, hydroxyethylenyl, dichloroethyl, piperidinylmethylenyl, tetrahydropyranylmethylenyl, cyclopropylmethylenyl, cyclohexylmethylenyl, or cyclopentylmethylenyl;
R⁶ is phenyl, phenyl-C₁-C₄-alkylenyl, thienyl, benzo[b]thienyl, benzo[b]furanyl, thieno[2,3-b]pyridinyl, thieno[3,2-b]thienyl, furo[3,2-b]pyridinyl, benzodiazinyl, imidazo[1,2-b]pyridazinyl, indolyl, thienyl-C₁-C₄-alkylenyl, cyclopenta[b]thienyl, C₃-C₇ cycloalkyl, C₃-C₇ cycloalkyl-C₁-C₄ alkylenyl, C₃-C₇ cycloalkyloxy-C₁-C₄ alkylenyl, C₃-C₇ cycloalkylamino, C₁-C₆-alkylamino, C₁-C₆-dialkylamino, N-ethenyl-tetrahydroindolyl, tetrahydroisoquinolinyl, phenylmethyltetrahydroisoquinolinyl, phenylcarbonyltetrahydroisoquinolinyl, or 1,1-dimethylethyldihydroisoquinolincarboxylate-yl, bicyclo[2.2.1]hept-2-yl-C₁-C₄ alkylenyl, wherein R⁶ is optionally substituted with one or more substituents selected from the group consisting of halo, C₁-C₄-alkyl, phenyl, halophenyl, and amino;
R⁷ is H or C₁-C₆ alkyl; and
R⁸ is H, C₁-C₆ alkyl, COOH, acetylamino-C₁-C₄ alkylenyl, or hydroxymethyl.

Thus, the present invention provides a method for activating a TRPV4 channel receptor in at least one cell expressing a TRPV4 channel receptor comprising contacting at least one cell with an effective amount of a pharmaceutical composition comprising an agonist to the said TRPV4 channel receptor. In one aspect, at least one cell is from a human. In another aspect, at least one cell is a chondrocyte. In another aspect, at least one cell is part of a cartilage matrix.

In another aspect, the agonist reduces the amount of matrix degrading enzymes produced by at least one cell. In another aspect, the agonist reduces the amount of matrix degrading enzymes released by at least one cell. In another aspect, the agonist reduces the amount of aggrecanase produced by at least one cell. In another aspect, the agonist reduces the amount of aggrecanase released by at least one cell. In another aspect, the agonist reduces the amount of matrix metalloproteases (“MMPs”) produced by at least one cell. In another aspect, the agonist reduces the amount of MMPs released by at least one cell. In another aspect, the MMPs are chosen from, but are not limited to, the group: MMP-1, MMP-3 and MMP-13.

In another aspect, the agonist reduces the amount of nitric oxide produced by at least one cell. In another aspect, the agonist reduces the amount of nitric oxide released by at least one cell.

In another aspect, the agonist attenuates inhibition of proteoglycan synthesis. In another aspect, the invention provides a method of increasing current flow through a TRPV4 channel receptor.

In another aspect, the agonist comprises 3-oxohexahydro-1H-azepin, azepine or acyclic 1,3-diamine. In another aspect, the agonist is chosen from the group of: 3-oxohexahydro-1H-azepin, azepine and acyclic 1,3-diamine. In another aspect of the present invention the agonist is not phorbol 12-myristate 13-acetate (PMA) or 4α-phorbol-12,13 didecanoate. 4α-phorbol-12,13 didecanoate has been shown to activate TRPV4 channel receptors. However, the potency and efficacy of 4α-phorbol-12,13 didecanoate in activating TRPV4 channel receptors is greatly diminished when 4α-phorbol-12,13 didecanoate is contacted with cartilage explants when compared with its efficacy and potency of contacting 4α-phorbol-12,13 didecanoate with chondrocytes that are extracted from a cartilage matrix. The compounds of the instant invention demonstrate potency and efficacy in activating TRPV4 channel receptors when contacted with human, rat, rabbit, mouse, canine, monkey, or bovine cartilage explants comprising chondrocytes.

In another aspect, the agonist comprises a 1,3-diamine. In another aspect, the agonist comprises an optionally substituted benzylsulfonamide. In another aspect, the agonist comprises a first chemical moiety selected from the group of: aryl or heteroaryl and a second chemical moiety which is an aryl optionally substituted with CN, NO₂, halogen, and wherein the first and second chemical moiety form part of the same compound. The agonist may further comprise a 1,3-diamine. In another aspect, the agonist comprises a first chemical moiety selected from the group of: benzothiophene, indenyl, or indole and a second chemical moiety selected from the group of: halogenated benzene or cyano-benzene. In another aspect, the agonist is chosen from the group of: N-{(1S)-1-[({(4R)-1-[(4-chlorophenyl)sulfonyl]-3-oxohexahydro-1H-azepin-4-yl}amino)carbonyl]-3-methylbutyl}-1-benzothiophene-2-carboxamide; N-{(1S)-1-[({(4R)-1-[(4-fluorophenyl)sulfonyl]-3-oxohexahydro-1H-azepin-4-yl}amino)carbonyl]-3-methylbutyl}-1-benzothiophene-2-carboxamide; N-{(1S)-1-[({(4R)-1-[(2-cyanophenyl)sulfonyl]-3-oxohexahydro-1H-azepin-4-yl}amino)carbonyl]-3-methylbutyl}-1-methyl-1H-indole-2-carboxamide; N-{(1S)-1-[({(4R)-1-[(2-cyanophenyl)sulfonyl]hexahydro-1H-azepin-4-yl}amino)carbonyl]-3-methylbutyl}-1-methyl-1H-indole-2-carboxamide; N-{(1S)-1-[({3-[[(cyanophenyl)sulfonyl](methyl)amino]propyl}amino)carbonyl]-3-methylbutyl}-1-benzothiophene-2-carboxamide; and N-{(1S)-1-[({3-[[(2,4-dichlorophenyl)sulfonyl](methyl)amino]propyl}amino)carbonyl]-3-methylbutyl}-1-benzothiophene-2-carboxamide.

In another aspect, the agonist has an EC50 value for TRPV4 channel receptor of less than about 1.0 μM. In another aspect, the agonist has an EC50 value for TRPV4 channel receptor of less than about 10 nM. In another aspect, the agonist inhibits GAG release induced by catabolic stimuli in articular cartilage explants with an IC₅₀ value of less than about 1.0 μM. In another aspect, the agonist has an EC50 value for TRPV4 channel receptor of less than about 1.0 μM as measured by calcium influx in isolated chondrocytes. In another aspect, the agonist increases current flow through said TRPV4 channel receptor.

In another aspect, the present invention provides a method for treating a patient in need thereof comprising contacting at least one cell expressing a TRPV4 channel receptor of the patient with a therapeutically effective amount of an agonist to a TRPV4 channel receptor. In one aspect, the patient is suffering from a disease of the cartilage. In another aspect, the patient is suffering from a disease or condition chosen from the group of: pain, chronic pain, neuropathic pain, postoperative pain, rheumatoid arthritis, osteoarthritis, neuralgia, neuropathies, algesia, nerve injury, ischaemia, neurodegeneration, cartilage degeneration, stroke, incontinence, inflammatory disorders, irritable bowel syndrome, obesity, periodontal disease, aberrant angiogenesis, tumor invasion and metastasis, corneal ulceration, and complications of diabetes. In another aspect, the patient suffers from a disease affecting the larynx, trachea, auditory canal, intervertebral discs, ligaments, tendons, joint capsules or bone development. In another aspect, the disease is related to joint destruction. In another aspect, the patient is suffering from osteoarthritis.

In another aspect, the agonist comprises 3-oxohexahydro-1H-azepin, azepine or acyclic 1,3-diamine. In another aspect, the agonist is chosen from the group of: 3-oxohexahydro-1H-azepin, azepine and acyclic 1,3, diamine. In another aspect, the agonist is chosen from the group of: N-{(1S)-1-[({(4R)-1-[(4-chlorophenyl)sulfonyl]-3-oxohexahydro-1H-azepin-4-yl}amino)carbonyl]-3-methylbutyl}-1-benzothiophene-2-carboxamide; N-{(1S)-1-[({(4R)-1-[(4-fluorophenyl)sulfonyl]-3-oxohexahydro-1H-azepin-4-yl}amino)carbonyl]-3-methylbutyl}-1-benzothiophene-2-carboxamide; N-{(1S)-1-[({(4R)-1-[(2-cyanophenyl)sulfonyl]-3-oxohexahydro-1H-azepin-4-yl}amino)carbonyl]-3-methylbutyl}-1-methyl-1H-indole-2-carboxamide; N-{(1S)-1-[({(4R)-1-[(2-cyanophenyl)sulfonyl]hexahydro-1H-azepin-4-yl}amino)carbonyl]-3-methylbutyl}-1-methyl-1H-indole-2-carboxamide; N-{(1S)-1-[({3-[[(cyanophenyl)sulfonyl](methyl)amino]propyl}amino)carbonyl]-3-methylbutyl}-1-benzothiophene-2-carboxamide; and N-{(1S)-1-[({3-[[(2,4-dichlorophenyl)sulfonyl](methyl)amino]propyl}amino)carbonyl]-3-methylbutyl}-1-benzothiophene-2-carboxamide.

In another aspect of the present invention, compounds are provided comprising a 1,3-diamine wherein a compound activates a TRPV4 channel receptor or a TRPV4 channel receptor variant when said compound is contacted with at least one cell expressing said TRPV4 channel receptor or said TRPV4 channel receptor variant. In another aspect, the compound comprises an optionally substituted benzylsulfonamide. In yet another aspect, the compound comprises a first chemical moiety selected from the group of: aryl or heteroaryl and a second chemical moiety which is an aryl optionally substituted with CN, NO₂, halogen, and wherein the first and second chemical moiety are comprised in the same compound. In another aspect, the compound comprises a first chemical moiety selected from the group of: benzothiophene, indenyl, or indole and a second chemical moiety selected from the group of: halogenated benzene or cyano-benzene.

In yet another aspect of the present invention, compounds are provided such that when a compound is contacted with at least one cell expressing at least one TRPV4 channel receptor or at least one TRPV4 channel receptor variant, the compound reduces an amount of at least one type of matrix degrading enzymes produced by said at least one cell. In another aspect, the compound reduces an amount of at least one type of matrix degrading enzymes produced or released by at least one cell expressing at least one TRPV4 channel receptor or at least one TRPV4 channel receptor variant. The compound may also reduce the amount of aggrecanase produced or released by a cell expressing at least one TRPV4 channel receptor or at least one TRPV4 channel receptor variant. Also provided are compounds that reduce an amount of at least one type of matrix metalloproteases produced or released by said at least one cell. Compounds of the present invention may also reduce the amount of nitric oxide produced or released by a cell expressing at least one TRPV4 channel receptor or at least one TRPV4 channel receptor variant. In another aspect, compound of the present invention may attenuate inhibition of proteoglycan synthesis in a cell expressing at least one TRPV4 channel receptor or at least one TRPV4 channel receptor variant.

In another aspect, treatment of the patient reduces the amount of aggrecan degradation in the patient. In another aspect, treatment of the patient reduces the amount of collagen degradation in the patient. In another aspect, treatment of the patient attenuates cartilage degradation, which may be in response to inflammatory mediators. In another aspect, cartilage degradation in the patient may be in response to injury.

In another aspect, a method is provided for attenuating decreased matrix protein production in the patient. In another aspect, the matrix protein is chosen from the group of: aggrecan and type II collagen. In another aspect, a method is provided for attenuating increased production of matrix degrading enzymes. In another aspect, the matrix degrading enzymes are chosen from, but are not limited to, the group of: MMP-1, MMP-3, MMP-9, MMP-13, ADAMTS4, and ADAMTS5. In another aspect, the production of matrix degrading enzymes is induced by inflammatory mediators. In another aspect, the production of matrix degrading enzymes is induced due to injury. In another aspect, a method is provided for reducing the amount of nitric oxide produced by at least one cell in cartilage. In another aspect, a method is provided for attenuating inhibition of proteoglycan synthesis.

TRPV4 channel receptor agonists of the present invention may be administered by any appropriate route. Suitable routes may include, but are not limited to, oral, rectal, nasal, topical (including, but not limited to, buccal and sublingual), vaginal, and parenteral (including, but not limited to, subcutaneous, intramuscular, intraveneous, intradermal, intrathecal, and epidural). It will be appreciated that a route of administration may vary with, for example, the condition of the recipient.

Pharmaceutical formulations adapted for oral administration may be presented as discrete units such as capsules or tablets; powders or granules; solutions or suspensions in aqueous or non-aqueous liquids; edible foams or whips; chewable gum; or oil-in-water liquid emulsions or water-in-oil liquid emulsions, among others.

For instance, for oral administration in the form of a tablet or capsule, an active drug component may be combined with an oral, non-toxic pharmaceutically acceptable inert carrier such as ethanol, glycerol, water and the like. Powders may be prepared by comminuting a compound to a suitable fine size and mixing with a similarly comminuted pharmaceutical carrier such as an edible carbohydrate, as, for example, starch or mannitol. Flavoring, preservative, dispersing and coloring agent may also be present.

Capsules may be made, for example, by preparing a powder mixture as described above, and filling formed gelatin sheaths. Glidants and lubricants such as colloidal silica, talc, magnesium stearate, calcium stearate or solid polyethylene glycol may be added to a powder mixture before a filling operation. A disintegrating or solubilizing agent such as agar-agar, calcium carbonate or sodium carbonate may also be added to improve the availability of a medicament when a capsule is ingested.

Moreover, when desired or necessary, suitable binders, lubricants, disintegrating agents and coloring agents may also be incorporated into a mixture. Suitable binders may include, but are not limited to, starch, gelatin, natural sugars such as glucose or beta-lactose, corn sweeteners, natural and synthetic gums such as acacia, tragacanth or sodium alginate, carboxymethylcellulose, polyethylene glycol, waxes and the like. Lubricants used in these dosage forms may include, but are not limited to, sodium oleate, sodium stearate, magnesium stearate, sodium benzoate, sodium acetate, sodium chloride and the like. Disintegrators may include, without limitation, starch, methyl cellulose, agar, bentonite, xanthan gum and the like. Tablets may be formulated, for example, by preparing a powder mixture, granulating or slugging, adding a lubricant and disintegrant and pressing into tablets. A powder mixture may be prepared by mixing a compound, suitably comminuted, with a diluent or base as described above, and optionally, with a binder such as carboxymethylcellulose, an aliginate, gelatin, or polyvinyl pyrrolidone, a solution retardant such as paraffin, a resorption accelerator such as a quaternary salt and/or an absorption agent such as bentonite, kaolin or dicalcium phosphate, among others. A powder mixture may be granulated by wetting with, for example, a binder such as syrup, starch paste, acadia mucilage or solutions of cellulosic or polymeric materials and forcing through a screen. As an alternative to granulating, a powder mixture may be run through a tablet machine forming imperfectly formed slugs broken into granules. Granules may be lubricated to prevent sticking to tablet-forming dies by means of addition of stearic acid, a stearate salt, talc or mineral oil, among others. A lubricated mixture may then be compressed into tablets. Compounds of the present invention may also be combined, for example, with free flowing inert carrier and compressed into tablets directly without going through granulating or slugging steps. A clear or opaque protective coating consisting of, for example, a sealing coat of shellac, a coating of sugar or polymeric material and a polish coating of wax may be provided. Dyestuffs or other compounds may be added to these coatings to distinguish different unit dosages.

Oral fluids such as solution, syrups and elixirs may be prepared in dosage unit form so that a given quantity contains a predetermined amount of the compound. Syrups may be prepared by dissolving a compound in a suitably flavored aqueous solution, while elixirs are prepared through the use of a non-toxic alcoholic vehicle. Suspensions may be formulated by dispersing a compound in a non-toxic vehicle. Solubilizers and emulsifiers such as ethoxylated isostearyl alcohols and polyoxy ethylene sorbitol ethers, preservatives, flavor additive such as peppermint oil or natural sweeteners or saccharin or other artificial sweeteners, and the like may also be added.

Where appropriate, dosage unit formulations for oral administration may be microencapsulated. A formulation may also be prepared to prolong or sustain release as for example by coating or embedding particulate material in polymers, wax or the like. In addition, oral formulation may be in the form of a chewable gum.

Pharmaceutical formulations adapted for transdermal administration may be presented as discrete patches intended to remain in intimate contact with the epidermis of the recipient for a prolonged period of time. For example, an active ingredient may be delivered from a patch by iontophoresis as generally described in Pharmaceutical Research, 3(6), 318 (1986).

Pharmaceutical formulations adapted for rectal administration may be presented as suppositories or as enemas, among others.

Pharmaceutical formulations adapted for parenteral administration may include, but are not limited to, aqueous and non-aqueous sterile injection solutions that may contain anti-oxidants, buffers, bacteriostats and solutes that render a formulation isotonic with the blood of an intended recipient; and aqueous and non-aqueous sterile suspensions that may include suspending agents and thickening agents. Formulations may be presented in unit-dose or multi-dose containers, for example sealed ampoules and vials, and may be stored in a freeze-dried (lyophilized) condition requiring only addition of a sterile liquid carrier, for example water for injections, immediately prior to use. Extemporaneous injection solutions and suspensions may be prepared from sterile powders, granules and tablets.

It should be understood that in addition to the ingredients particularly mentioned above, formulations may include, but are not limited to, other agents conventional in the art having regard to the type of formulation in question, for example those suitable for oral administration may include flavoring agents.

Salts encompassed within the term “pharmaceutically acceptable salts” refer to non-toxic salts of the compounds of this invention that are prepared, for example, by reacting a free base with a suitable organic or inorganic acid or by reacting an acid with a suitable organic or inorganic base.

Typically, a therapeutically effective amount of a TRPV4 channel receptor agonist of the present invention will depend upon a number of factors including, for example, age and weight of a mammal, at least one precise condition requiring treatment, severity of a condition, nature of a formulation, and route of administration. Ultimately, a therapeutically effective amount will be at the discretion of an attendant physician or veterinarian.

### EXAMPLES

The following examples illustrate various aspects of this invention. These examples do not limit the scope of this invention which is defined by the appended claims.

### Example 1

### TRPV4 Channel Receptor is Expressed in Cartilage and Chondrocytes

Tissue and cell expression of human TRPV4 channel receptor was studied using TaqMan (Perkin Elmer) quantitative RT-PCR (Gibson et al., 1996) according to the manufacturer's instructions. TaqMan reactions were conducted using probes for human GAPDH, cyclophilin and human TRPV4 channel receptor. The human TRPV4 channel receptor probe consisted of:

5′-ATGAGGACCAGACCAACTGCA; and (SEQ ID NO:3)

5′-GGAGGAAGGTGCTGAAGGTCTC flanking primers and a (SEQ ID NO:4)

5′-CACTTACCCCTCGTGCCGTGACAG fluorogenic probe. (SEQ ID NO:5)

Data were analysed using the Power Macintosh software accompanying the ABI Prism™ 7700.

The data from a screen of body tissues, shown in Table 1, shows that human TRPV4 channel receptor is most prominently expressed in cartilage. A screen of primary and clonal cell cultures shows significant expression only in chondrocytes.

TABLE 1
Relative mRNA expression in human tissues and cell-lines.

| | A | B | C | D | E | F | G | H | I | J | K | L | M | N | O | P | Q | R | S | T |
|---|---|---|---|---|---|---|---|---|---|---|---|---|---|---|---|---|---|---|---|---|
| T* | 0 | 1 | 0 | 1 | 1 | 1 | 2 | 0 | 0 | 0 | 1 | 0 | 0 | 1 | 1 | 1 | 2 | 5 | 1 | 0 |
| C** | 0 | 0 | 0 | 0 | 0 | 0 | 0 | 0 | 0 | 0 | 0 | 0 | 0 | 0 | 0 | 0 | 0 | 5 | 0 | 0 |

VR4 Expression (copies/ng mRNA)

| | |
|---|---|
| For T | For C |
| 0 < 500 | 0 < 1000 |
| 1 = 500 to 2000 | 5 = 40K to 50K |
| 2 = 2000 to 6000 | |
| 5 = 20K to 30K | |

*T relates to the category of different body tissues as follows:

| | | |
|---|---|---|
| A = CNS | B = pituitary | C = heart |
| D = lung | E = liver | F = foetal liver |
| G = kidney | H = skeletal | I = stomach |
| | muscle | |
| J = intestine | K = spleen | L = lymphocytes |
| M = macrophages | N = adipose | O = pancreas |
| P = prostate | Q = placenta | R = cartilage |
| S = bone | T = bone marrow | |

**C relates to the category of different cell lines as follows:

| | | |
|---|---|---|
| A = aortic smooth muscle cells | B = bladder | |
| | smooth | |
| | muscle cells | |
| C = C20A4 | D = MG63 | E = SAOS2 |
| F = lymphocyte | G = macrophage | H = platelets |
| I = neutrophil | J = CHANG | K = HepG2 |
| L = IMR32 | M = SK-N-MC | N = SK-N-SH |
| O = NT-2 | P = 1321N1 | Q = C13 |
| R = primary human chondrocytes | S = Hs-683 | |
| T = HEK293. | | |

The results show high-levels of expression in cartilage tissue (T:R) and primary human chondrocytes (C:R).

### Example 2

### TRPV4 Channel Receptor is Activated by 4α-phorbol-12,13 didecanoate (4α-PDD)

TRPV4 channel receptor cDNA was inserted into the expression vector pcDNA3.1 V5-His (Invitrogen, Carlsbad, Calif.). Wildtype HEK293 cells, or HEK293 cells transfected with the human TRPV4 channel receptor: pcDNA3.1 V5-His construct, or mock transfected cells, or bovine chondrocytes, were seeded into 96-well microtitre plates at 25,000 cells/well and cultured overnight. The cells were then incubated with 4 μM Fluo-3 (Fluo-3: Molecular Probes (Eugene, Oreg.)) for 2 hours at room temperature in the dark. Dye loaded cells were washed 4× with Tyrodes buffer: (NaCl, 145 mM; KCl, 2.5 mM; Hepes, 10 mM; Glucose, 10 mM; MgCl₂, 1.2 mM; CaCl₂, 1.5 mM), which also contained 0.2% BSA but not probenecid. Agonists and antagonists were also prepared in Tyrodes buffer. Cells were preincubated for 30 minutes with antagonist or buffer. Agonist addition and measurement of cytoplasmic calcium concentration was performed in the FLIPR (Smart, et al., (2000) Br. J. Pharmacol. 129, 227-230).

Both phorbol 12-myristate 13-acetate (PMA) and 4α-phorbol-12,13-didecanoate (4αPDD) increased intracellular calcium in HEK293-TRPV4 channel receptor cells (Table 2) but were without effect in wild type HEK293 cells or in cells transfected with empty vector. PMA also activated VR1, but was only a partial agonist (Emax 0.46) compared to capsaicin and RTX. 4αPDD did not activate VR1 (Table 2). In conclusion, 4×PDD acts as a TRPV4 channel receptor selective agonist.

TABLE 2
PMA and 4αPDD Intracellular Calcium Production in HEK293-TRPV4
Channel Receptor Cells

| | pEC50 | | | |
|---|---|---|---|---|
| | wild type | empty vector | TRPVR1 | TRPVR4 |
| RTX | IA | IA | 8.93 ± 0.20 | IA |
| capsaicin | IA | IA | 7.48 ± 0.12 | IA |
| PMA | IA | IA | 7.86 ± 0.06 | 6.64 ± 0.06 |
| 4□PDD | IA | IA | IA | 5.73 ± 0.06 |

Data are mean ± s.e.mean, where n = 3-5. IA = inactive

Bovine articular chondrocytes responded to 4α-PDD with a similar dose dependency as the transfected BEK293 cells. The response to 4α-PDD had a similar kinetic profile and concentration dependency to that seen for the recombinant TRPV4 channel receptor expressed in HEK293 cells. The response was dependent upon extracellular calcium ions and was blocked by the channel blocker, ruthenium red. These data suggest that the response to 4α-PDD was due to a TRPV4 channel receptor endogenously expressed by chondrocytes.

### Example 3

### Ca²⁺ Mobilization in Primary Chondrocytes (Human, Bovine, Rat)

TRPV4 channel receptor is a Ca²⁺ permeable, non-selective, ligand-gated cation channel. Ca²⁺ influx mediated through TRPV4 channel receptors was measured in human, rat and bovine chondrocytes using standard techniques in the art (e.g. employing a FlexStation manufactured by Molecular Devices (Sunnyvale, Calif.)). 4□-PDD, a known agonist to TRPV4 channel receptor, stimulated Ca²⁺ influx in chondrocytes from all three species, while PMA and capscaicin, known agonists to VR1, produced no change in Ca²⁺ influx. In addition, Ruthenium Red, a known inhibitor of 4□-PDD was found to reverse the effects of 4□-PDD on chondrocytes from all species and reduce Ca²⁺ influx down to baseline levels. (Watanabe, et al. (2002). J. Biol. Chem. 277(16): 13569-47051.).

### Example 4

### Preparation of N-{(1S)-1-[({(4R)-1-[(2-cyanophenyl)sulfonyl]hexahydro-1H-azepin-4-yl}amino)carbonyl]-3-methylbutyl}-methyl-1H-indole-2-carboxamide—(Formula Ib)

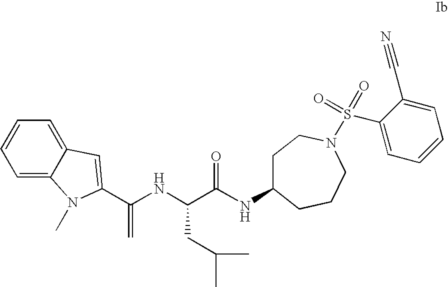

Formula Ib was prepared as follows:

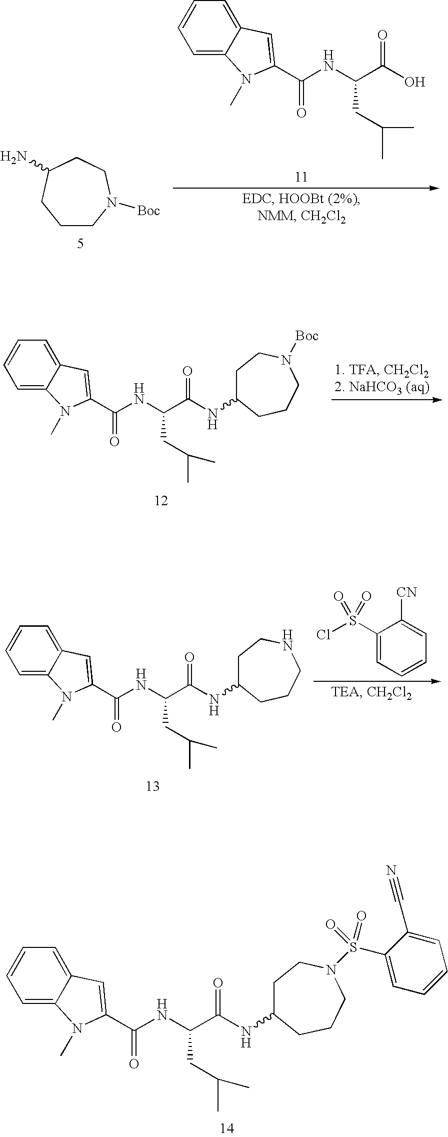

To a CH₂Cl₂ (25 mL, 0.2 M) solution of 4-amino azepine 5 (1.03 g, 4.8 mmol) and ((S)-4-Methyl-2-{[1-(1-methyl-1H-indol-2-yl)-methanoyl]-amino}-pentanoic acid 11 (1.52 g, 5.29 mmol) were added EDC HCl (1.11 g, 5.78 mmol), 3-hydroxy-1,2,3-benzotriazin-4(3H)-one (HOOBt; 0.16 g, 0.096 mmol), and N-methyl-morpholine (NMM; 0.58 mL, 5.29 mmol). This solution was allowed to stir overnight at room temperature. The reaction mixture washed with 1N HCl and brine, and the organic portion was dried over MgSO₄, filtered, and concentrated to 2.5 g of a yellow solid. This material was combined with material from a previous reaction (3.5 g combined weight) and purified by FCC (SiO₂, 2% CH₃OH/CH₂Cl₂). The title compound was obtained as a yellow solid (3 g).

To a CH₂Cl₂ solution (150 mL, 0.02M) of the Boc-protected amine 12 (1.5 g, 3.1 mmol) from the previous Example was added trifluoroacetic acid (2 mL, 6.5 mmol), and the solution was stirred at room temperature for 1 h. The reaction mixture was then concentrated in vacuo, redissolved in CH₃CN (50 mL) and concentrated again. The residue was then dissolved in CH₂Cl₂ and washed with one portion of 5% NaHCO₃. The remaining organic layer was dried (MgSO₄), filtered, and concentrated to give the title compound as a yellow solid. This material was carried on to the next reaction without further purification.

To a CH₂Cl₂ solution (5 mL, 0.15 M) of the free amine 13 (0.30 g, 0.78 mmol) was added 2-cyanobenzenesulfonyl chloride (0.16 g, 1.56 mmol) and triethyl amine (0.33 mL, 2.34 mmol). The mixture, eventually becoming a brown solution, was stirred at room temperature for 3 h. The reaction mixture was purified directly by FCC (SiO₂) and eluted with 20% CH₂Cl₂/ethyl acetate to provide 0.29 g of a yellow solid (68%).

¹H NMR (400 MHz, CDCl₃) (mixture of two diastereomers): δ 8.05 (m, 1H); 7.85 (m, 1H); 7.72-7.60 (m, 3H); 7.38-7.30 (m, 2H); 7.15 (m, 1H), 6.84 (m, 1H); 6.60 (m, 1H); 4.65 (m, 1H); 4.14 (m, 1H), 4.03 (m, 3H); 3.75-3.61 (m, 2H); 3.27 (m, 1H); 3.20-3.04 (m, 1H); 2.14-1.69 (m, 9H), 1.01 (m, 6H).

LCMS (M+H): 550.

A diasteraomerically pure product was obtained using the following methods:

The individual C4 diastereomers were separated by high pressure liquid chromatography with a 10 u Chiralcel OD column (20×250 mm) (J. T. Baker, Phillipsburg, N.J.) with 20% ethanol/hexanes as the eluent (isocratic, flow rate of 15 mL/min). Upon isolation the stereochemistry of the individual analogs were determined by a combination of virtual circular dichroism (VCD) and small molecule X-ray crystallography.

### Example 5

### Preparation of N-{(1S)-1-[({(4R)-1-[(4-fluorophenyl)sulfonyl]-3-oxohexahydro-1H-azepin-4-yl}amino)carbonyl]-3-methylbutyl}-1-benzothiophene-2-carboxamide (Formula IIb)

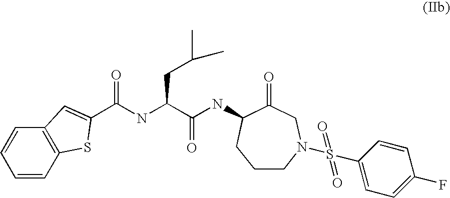

Formula IIb was prepared as follows:

### a.) {(S)-1-[3-Hydroxy-1-(4-fluorobenzenesulfonyl)-azepan-4-ylcarbamoyl]-3-methyl-butyl}-carbamic acid-tert-butyl ester

{(S)-1-[3-Hydroxy-1-(-(4-fluorobenzenesulfonyl)-azepan-4-ylcarbamoyl]-3-methyl-butyl}-carbamic acid-tert-butyl ester (0.8 g, 2.33 mmol) was dissolved in 1,2-dichloroethane (DCE, 20 ml). Then, morpholinemethyl polystyrene resin beads (1.26 g, 3.7 mmol/g, Nova Chemicals (Alberta, Canada)) were added and the solution was shaken for 5 minutes. Then, p-fluorobenzenesulfonyl (0.48 g, 2.33 mmol) was dissolved in DCE (10 ml), and this solution was added to the reaction mixture. The reaction was shaken overnight, filtered, washed with DCE (2×10 ml), then CH₂Cl₂ (10 ml). The combined organics were concentrated in vacuo, and used in the next reaction without further purification: M+H⁺=514.2.

### b.) (S)-2-Amino-4-methyl-pentanoic acid [3-hydroxy-1-(4-fluorobenzenesulfonyl)-azepan-4-yl]-amide-HCl salt

{(S)-1-[3-Hydroxy-1-(-(4-fluorobenzenesulfonyl)-azepan-4-ylcarbamoyl]-3-methyl-butyl}-carbamic acid-tert-butyl ester (0.59 g, 1.15 mmol) was dissolved in CH₂Cl₂ (8 ml), then a solution of 4 M HCl in dioxane (8 ml) was added and the reaction was stirred at RT for 4 hours. The reaction mixture was concentrated in vacuo, azeotroped from toluene twice (10 ml) in vacuo, and was used in the next reaction without further purification: M+H⁺=413.8.

### c.) (S)-2-(2-Benzo[b]thiophene-2-carboxylic acid)-4-methyl-pentanoic acid [3-hydroxy-1-(-(4-fluorobenzenesulfonyl)-azepan-4-yl]-amide

(S)-2-Amino-4-methyl-pentanoic acid [3-hydroxy-1-(4-fluorobenzenesulfonyl)-azepan-4-yl]-amide-HCl salt was dissolved in MeOH (10 ml) and was treated with carbonate-polystyrene resin beads (1.75 g, 2.63 mmol/g, 4.6 mmol) and was shaken for 2 hours, filtered, washed with MeOH (10 ml) and the combined organics were concentrated in vacuo. The product was then dissolved in DCE (2 ml) and morpholinemethyl polystyrene resin beads (0.25 g, 3.77 mmol/g, 0.91 mmol, Nova) were added and the reaction was shaken for 5 minutes. Then, benzo[b]thiophenecarbonyl chloride (0.44 mmol) was added and the reaction mixture was shaken overnight. Then, trisamine polystyrene beads (0.1 g, 3.66 mmol/g, 0.366 mmol) was added and the reaction mixture was shaken for 1.5 h. The reaction mixture was then filtered, washed with DCE (2×10 ml) and CH₂Cl₂ (10 ml), and the combined organics were concentrated in vacuo. The crude product was used in the next reaction without further purification: M+H⁺=562.2.

### d.) N-{(1s)-1-[({(4r)-1-[(4-fluorophenyl)sulfonyl]-3-oxohexahydro-1 h-azepin-4-yl}amino)carbonyl]-3-methylbutyl}-1-benzothiophene-2-carboxamide

(S)-2-(2-Benzo[b]thiophene-2-carboxylic acid)-4-methyl-pentanoic acid [3-hydroxy-1-(4-fluorobenzenesulfonyl)-azepan-4-yl]-amide (0.24 g, 0.44 mmol) was dissolved in CH₂Cl₂ (5 ml), then Dess-Martin periodinane (0.3 g, 0.7 mmol) was added and the reaction was stirred for 30 minutes. The reaction was diluted with CH₂Cl₂ (20 ml), then was extracted with aqueous 10% Na₂S₂O₅ (10 ml), then aqueous 10% NaHCO₃ (10 ml), water (10 ml), brine (10 ml). The combined organics were concentrated in vacuo.

The residue was purified by HPLC. First eluting diastereomer: ms 560.2 (M+H⁺). ¹H NMR (500 MHz; CDCl₃): δ 7.80-7.72 (m, 5h) 7.37-7.34 (m, 2h), 7.33-7.15 (m, 4h), 2.43 (t, 1 h), 0.96 (d, 6h). Second eluting diastereomer: ms 560.2 (M+H⁺).

### Example 6

### Preparation of N-{(1S)-1-[({(4R)-1-[(4-chlorophenyl)sulfonyl]-3-oxohexahydro-1H-azepin-4-yl}amino)carbonyl]-3-methylbutyl}-1-benzothiophene-2-carboxamide (Formula IIc)

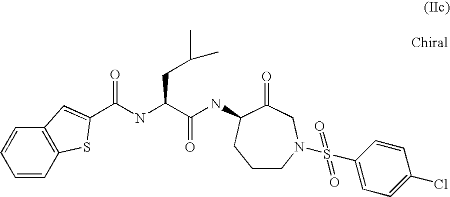

Formula IIc was prepared as follows:

### a.) {(S)-1-[1-(3-Chloro-benzenesulfonyl)-3-hydroxy-azepan-4-ylcarbamoyl]-3-methyl-butyl}-carbamic acid tert-butyl ester

To a solution of the compound [(S)-1-(3-Hydroxy-azepan-4-ylcarbamoyl)-3-methyl-butyl]-carbamic acid tert butyl ester (2.50 g, 7.29 mmol) in DCE (100 ml) was added P-NMM (4.0 g) and 4-chlorobenzenesulphonyl chloride (1.85 g, 8.75 mmol). After shaking at room temperature for over night, the solution was filtered. The filtrate was concentrated to yield the title compound as white solid (3.13 g, 83.3%). MS: 539.78 (M+Na)⁺.

### b.) (S)-2-Amino-4-methyl-pentanoic acid [1-(3-chloro-benzenesulfonyl)-3-hydroxy-azepan-4-yl]-amide

To a stirring solution of the compound of Example 6a (1.0 g, 1.93 mmol) in methanol (10 ml) was added HCl (4M in dioxane) (10 ml). After stirring at room temperature for 3 hours, the solution was concentrated to provide a white solid. To a solution of the white solid (0.68 g, 1.50 mmol, 78%) in methnol (37 ml) was added P—CO₃ (2.85 g, 2.63 mmol/g). After shaking for 2 hours, the solution was filtered and concentrated to yield the title compound as white solid (0.59 g, 1.42 mmol, 95%): MS: 417.86 (M+H)⁺.

### c.) Benzo[b]thiophene-2-carboxylic acid-{(S)-1-[1-(4-chloro-benzenesulphonyl)-3-hydroxy-azepan-4-ylcarbamoyl]-3-methyl-butyl}-amide

To a solution of the compound of Example 6b (0.14 g, 0.335 mmol) in CH₂Cl₂ (20 mL) was added benzo[b]thiophene-2-carboxylic acid (0.81, 0.50 mmol), 1-hydroxybenzotriazole (0.77 g, 0.569 mmol), and P-EDC (0.67 g, 1 mmol/g) in CH₂Cl₂ (10 mL). After shaking at room temperature overnight, the solution was treated with tisamine (0.446 g, 3.75 mmol/g). After shaking for another 2 hours, the solution was filtered and concentrated to yield the title compound as a white solid (122.2 mg, 65%). MS (ESI): 562.2 (M+H)⁺.

### d.) N-{(1S)-1-[({(4R)-1-[(4-chlorophenyl)sulfonyl]-3-oxohexahydro-1H-azepin-4-yl}amino)carbonyl]-3-methylbutyl}-1-benzothiophene-2-carboxamide

To a stirring solution of the compound of Example 6c (122.2 mg, 0.217 mmol) in dichloromethane (4 mL) was added Dess-Martin reagent (184.8 mg, 0.436 mmol). After stirring at room temperature for 2 hours, solutions of sodium thiosulfate (2 mL of 10% in water) and saturated aqueous sodium bicarbonate (2 mL) were added simultaneously to the solution. The aqueous was extracted with dichloromethane (2×). The organic phases were combined, washed with saturated brine, dried (MgSO₄), filtered and concentrated. The residue was purified by HPLC to give the first eluting diastereomer as a white solid (41 mg, 33%): MS (ESI) 576.2 (M+H)⁺ and the second eluting diastereoemer as a white solid (32.6 mg, 26%): MS (ESI) 576.2 (M+H)⁺

### Example 7

### Preparation of N-{(1s)-1-[({(4r)-1-[(2-Cyanophenyl)Sulfonyl]-3-Oxohexahydro-1H-Azepin-4-Yl}Amino)Carbonyl]-3-Methylbutyl}-1-Benzothiophene-2-Carboxamide—Formula IId

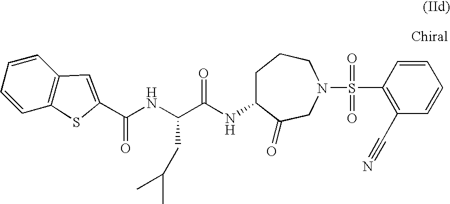

Formula IId was prepared as detailed in Example 6 except substituting 2-cyanobenzenesulphonyl chloride for 4-chlorobenzenesulfonyl chloride in step 6a.

### Example 8

### Preparation of N-{(1S)-1-[({3-[[(cyanophenyl)sulfonyl](methyl)amino]propyl}amino)carbonyl]-3-methylbutyl}-1-benzothiophene-2-carboxamide—Formula IIIb

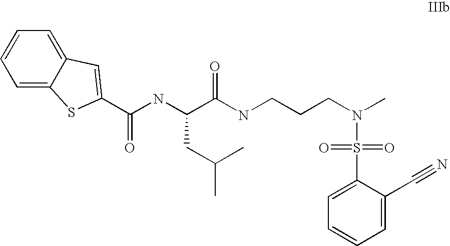

Formula IIIb was prepared as follows:

### a. N-(3-aminopropyl)-2-cyano-N-methylbenzenesulfonamide

To a solution of N-methylethylene diamine (100 mg, 1.35 mmol) and 2-cyanobenzenesulfonyl chloride (250 mg, 1.23 mmol) in CH₂Cl₂ (5 mL) was added Et₃N (0.19 mL, 1.35 mmol) at 0° C. under N₂. The reaction mixture was warmed up to RT and stirred overnight. Additional CH₂Cl₂ was added to the mixture. This mixture washed with brine. After drying over MgSO₄, filtration, and evaporation under reduced pressure provided the crude material (226 mg).

### b. 1-[(1-benzothien-2-ylcarbonyl)oxy]-2,5-pyrrolidinedione

A dry 1.0 L round bottom flask was charged with methylene chloride (281 mL), 1-benzothiophene-2-carboxylic acid (10 g, 56.18 mmol), N-hydroxysuccinimide (7.11 g, 61.8 mmol), and 1-[3-(dimethylamino)propyl]-3-ethylcarbodimide hydrochloride (12.92 g, 67.40 mmol), and the reaction mixture was stirred under nitrogen at room temperature (RT) for 4 hrs. The solvent was partially removed under reduced pressure and the residue washed with brine (2×). The organic solution was dried over MgSO₄ and concentrated. The obtained white solid product (15.4 g) was carried on to the next step without further purification.

### c. N-(1-benzothien-2-ylcarbonyl)-L-leucine

A dry 1.0 L round bottom flask was charged with 1-[(1-benzothien-2-ylcarbonyl)oxy]-2,5-pyrrolidinedione (15.4 g, 56.18 mmol), L-leucine (7.66 g, 58.43 mmol), EtOH (140 mL), methylene chloride (85 mL) and deionized water (55 mL). The reaction mixture was cooled to 5-10° C. with an ice-water bath, whereupon triethylamine (9.4 mL, 67.42 mmol) was added slowly. The ice water bath was removed and the mixture was stirred at ambient temperature overnight. The following morning the mixture was diluted with 50 mL water and the pH of the aqueous layer adjusted to 1 with 6N HCl. This mixture was then extracted with methylene chloride (2×) and the organic layer dried over MgSO₄ and concentrated to afford the product (16.4 g) as a white solid.

### d. N-{(1S)-1-[({3-[[(2-cyanophenyl)sulfonyl](methyl)amino]-propyl}amino)carbonyl]-3-methylbutyl}-1-benzothiophene-2-carboxamide

To a solution of N-(3-aminopropyl)-2-cyano-N-methylbenzenesulfonamide (124 mg, 0.488 mmol) in CH₂Cl₂ was added N-(1-benzothien-2-ylcarbonyl)-L-leucine (142 mg, 0.488 mmol), followed by HOOBt (2.0 mg, 0.012 mmol). The mixture was cooled to 0° C. and N-methylmorpholine (0.081 mL, 0.732 mmol) was added. The mixture was stirred several minutes whereupon EDC HCl (103 mg, 0.537 mmol) was added. The mixture was allowed to warm to room temperature where it was maintained for an additional 3 hrs. The mixture was then washed with 10% citric acid aqueous solution, saturated NaHCO₃ and brine. The organic layer was dried over MgSO₄ and concentrated. The residue was purified by biotage chromatography (0% to 6% THF/DCM) to provide 133 mg of the title compound (52%); ¹H NMR (CDCl₃): δ 8.01-8.08(d, 1H), 7.65-7.90(m, 6H), 7.35-7.48(m, 2H), 6.76-6.90(m, 2H), 4.64-4.75(m, 1H), 3.51-3.62(m, 1H), 3.28-3.46(m, 2H), 3.10-3.16(m, 1H), 2.89(s, 3H), 1.68-1.95(m, 5H), 1.00(d, 6H); MS (m/z): 527.4(M+H).

### Example 9

### Preparation of N-{(1S)-1-[({3-[[(2,4-dichlorophenyl)sulfonyl](methyl)amino]propyl}amino)carbonyl]-3-methylbutyl}-1-benzothiophene-2-carboxamide—Formula IIIc

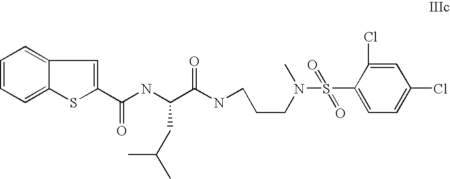

Formula IIIc was prepared as follows:

### a. N-(3-aminopropyl)-N-methyl-2-nitrobenzenesulfonamide

To the solution of N-methyl-1,3-propanediamine (2.54 g, 28.8 mmol) and 2-nitrobenzenesulfonyl chloride (4.26 g, 19.2 mmol) was added triethylamine (TEA, 5.35 mL, 38.4 mmol); the reaction mixture was stirred at RT for 3 hrs. Solvents were removed and pH was adjusted to 2.0-2.5 by 6N HCl; the organic material was extracted with dichloromethane (DCM) twice, followed by adjustment of the aqueous layer to pH ˜10. The aqueous layer was extracted with CHCl₃ five times. The collected solvent containing the extracted material was dried, filtered and concentrated to provide 4.6 g of a yellowish oil. The crude material was used directly to next step without further purification.

### b. N-((1S)-3-methyl-1-{[(3-{methyl[(2-nitrophenyl)sulfonyl]amino}propyl)-amino]carbonyl}butyl)-1-benzothiophene-2-carboxamide

To the solution of N-(3-aminopropyl)-N-methyl-2-nitrobenzene-sulfonamide (1.84 g, 6.74 mmol) of Example 2a in CH₂Cl₂ was added N-(1-benzothien-2-ylcarbonyl)-L-leucine (1.96 g, 6.74 mmol), followed by HOOBt (27.5 mg, 0.168 mmol). The mixture was cooled to 0° C. whereupon N-methylmorpholine (1.48 mL, 13.46 mmol) was added. The mixture was stirred several minutes whereupon EDC.HCl (1.29 g, 6.73 mmol) was added. The mixture was allowed to warm to room temperature where it was maintained for an additional 3 hrs. The mixture was then washed with 10% citric acid aqueous solution, saturated NaHCO₃ and brine. The organic layer was dried over MgSO₄ and concentrated. Purification of the residue by biotage chromatography (0%-10% THF/DCM) provided 1.49 g of the title compound (40.5%).

### c. N-[(1S)-3-methyl-1-({[3-(methylamino)propyl]amino}carbonyl)butyl]-1-benzothiophene-2-carboxamide

To the solution of the compound of Example 9b (834 mg, 1.53 mmol) in DMF was added benzenethiol (0.235 mL, 2.29 mmol) and K₂CO₃ (633 mg, 4.58 mmol). The reaction mixture was stirred at RT for 4 hrs, whereupon the solvent was removed and the residue diluted with 3 mL water and acidified to pH 1.5 with 1N HCl. This mixture was extracted with dichloromethane and the combined organic layers were washed with 1N HCl five times. The aqueous layers were combined and adjusted to pH 12.5, then extracted with ethyl acetate three times, then dried over K₂CO₃, filtered, and concentrated by rotary evaporation to give 420 mg title compound.

### d. N-{(1S)-1-[({3-[[(2,4-dichlorophenyl)sulfonyl](methyl)amino]-propyl}amino)carbonyl]-3-methylbutyl}-1-benzothiophene-2-carboxamide

To a solution of the compound of Example 9c (50 mg, 0.139 mmol) in dichloromethane was added triethylamine (0.042 mL, 0.305 mmol) and 2,4-dichlorobenzenesulfonyl chloride (37.3 mg, 0.152 mmol). The reaction mixture was stirred at RT for 2 hrs then washed with 10% aqueous citric acid, followed by washings with saturated NaHCO₃ and brine. The organic layer was dried over MgSO₄ and concentrated. Purification of the residue by biotage chromatography (0% to 8.0% THF/DCM) provided 71 mg of the title compound (89%); ¹H NMR (CDCl₃): δ 7.21-7.98 (m, 8H), 6.72-6.89(m, 1H), 6.62-6.68(m, 1H), 4.65-4.75(m, 1H), 3.25-3.46(m, 4H), 2.86(s, 3H), 1.68-1.95(m, 5H), 1.06(d, 6H); MS (m/z): 571.2(M+H).

### Example 10

### Reduction of IL-1 Induced MMP Production in Chondrocytes

IL-1 is a cytokine that can be used to stimulate chondrocytes. Exposure of chondrocytes to IL-1 increases the production of matrix metalloproteinases, including MMP-1, MMP-3, MMP-9, and MMP-13.

A concentration-dependent inhibition was observed for Formula IIb on IL-1 induced MMP-3 and MMP-13 production from human chondrocytes. Human chondrocytes were exposed to L-1 (10 ng/mL) to induce MMP-3 and MMP-13 production. Increased production of MMP-3 and MMP-13 was verified by both Northern blot (mRNA) and ELISA assay (protein). Cells were exposed to Formula IIb at concentrations ranging from 0.1 μM to 10 μM. As shown in Table 3, Formula IIb reduced the amount of MMP-3 and MMP-13 produced by chondrocytes exposed to IL-1. Similar results were confirmed by ELISA assay. TRPV4 channel receptor agonist inhibits IL-1-induced MMP-3 and MMP-13 production.

Table 3 presents IL-1 induced mRNA (mean ±SE) production for MMP-3 and MMP-13 in the presence and absence of agonist.

TABLE 3
Inhibition of IL-1 induced Human Articular Chondrocyte MMP mRNA
Expression (Fraction of IL-1; Mean ± SEM) After 24 Hours of Agonist
Exposure

| | mRNA expression (Fraction of IL-1) | | | |
|---|---|---|---|---|
| | (Mean ± SE) after | | | |
| | 24 hours of Agonist Exposure | | | |
| | MMP-3 | | MMP-13 | |
| Agonist Concentration (μM) | Mean | SEM | Mean | SEM |
| Control* | 0 | | 0 | |
| IL-1 (10.0 ng/mL)** | 1.0 | | 1.0 | |
| IL-1 + Formula IIb (0.1 μM) | 0.92 | 0.08 | 0.89 | 0.08 |
| IL-1 + Formula IIb (0.3 μM) | 0.91 | 0.01 | 0.87 | 0.02 |
| IL-1 + Formula IIb (1.0 μM) | 0.88 | 0.07 | 0.79 | 0.06 |
| IL-1 + Formula IIb (3.0 μM) | 0.68 | 0.10 | 0.29 | 0.08 |
| IL-1 + Formula IIb (10.00 μM) | 0.43 | 0.01 | 0.09 | 0.02 |

*no IL-1 or agonist
**10 ng/mL of IL-1 no agonist

### Example 11

### Reduction of IL-1 Induced Aggrecanase Production by Bovine Articular Chondrocytes

Aggrecanases (ADAMTSs), in conjunction with MMPs, degrade aggrecan, one of the major matrix components in articular cartilage. In cartilage explant cultures, IL-1 has been shown to induce aggrecanase production and activity, causing an accelerated degradation of the extracellular matrix [Arne, et al., J. Biol. Chem. 1999; 274(10):6594-6601]. Aggrecanase production is also induced in chondrocyte cultures by exposure to IL-1, and the resulting aggrecanase induction was measured using a chondrocyte-based aggrecanase peptide cleavage assay [Pratta et al; Arthritis Rheum. 2003; 48(1): 119-133]. After a 24 hour incubation, IL-1 significantly induced aggrecanase activity in bovine articular chondrocyte cultures. Incubation of the chondrocytes with IL-1 in combination with Formula IIb resulted in a concentration-dependent inhibition of the measured aggrecanase activity as shown in Table 4.

Formula IIb did not appear to inhibit peptide cleavage through direct inhibition of enzyme activity, since it had no effect on the cleavage of the peptide substrate by soluble ADAMTS-4 (aggrecanase-1).

Table 4 presents the effects of a TRPV4 channel receptor agonist on reducing IL-1 induced aggrecanase production (mean ±SE) in bovine articular chondrocytes.

TABLE 4
Reduction of IL-1 Induced Aggrecanase Production (Mean ± SEM)
by a TRPV4 Channel Receptor Agonist in Bovine Articular Chondrocytes.

| | Aggrecanase Production | |
|---|---|---|
| | (Mean ± SEM | |
| Treatment | OD 450 nm) | % Inhibition |
| control* | 0.573 ± 0.067 | — |
| IL-1 (3.0 ng/mL)** | 2.201 ± 0.132 | 0 |
| IL-1 + Formula IIb (0.03 μM) | 2.018 ± 0.044 | 11 |
| IL-1 + Formula IIb (0.10 μM) | 1.669 ± 0.029 | 33 |
| IL-1 + Formula IIb (0.30 μM) | 1.139 ± 0.032 | 65 |
| IL-1 + Formula IIb (1.00 μM) | 0.851 ± 0.024 | 83 |

*no IL-1 or agonist
**IL-1 alone no agonist

### Example 12

### Reduction of IL-1 Induced Aggrecan Degradation in Articular Cartilage

Articular cartilage explants isolated from newborn calves at slaughter were placed in culture, and aggrecan degradation was initiated through stimulation with IL-1. Since aggrecan degradation in this model is believed to be mediated by aggrecanase, TRPV4 channel receptor agonists that are active in the chondrocyte-based aggrecanase peptide assay (Example 11) may also be effective inhibitors in the cartilage explant assay, presumably by blocking aggrecanase production in these tissues.

Bovine articular cartilage explants were exposed to IL-1 alone and in combination with Formula IIc for 3 days. At the end of the culture period, conditioned media were assayed for glycosaminoglycan (GAG) levels by colorimetric (DMMB) assay as a measure of aggrecan degradation. In bovine explants, IL-1 induced a profound increase in the amount of GAG released into the media during the 3 day culture. When cartilage was treated with IL-1 in combination with Formula IIc, there was a concentration-dependent reduction in GAG release as shown in Table 5.

There was significant aggrecan protection by Formula IIc after a 3 day exposure to IL-1.

Significant inhibition by Formula IIc was observed even after IL-1 stimulation for 12 or 21 days. Table 5 presents the effects of TRPV4 channel receptor agonist on reducing IL-1 induced GAG release (mean ±SEM) in articular cartilage.

TABLE 5
Reduction of IL-1 Induced GAG Release (Mean ± SEM) in Articular
Cartilage Treated with a TRPV4 Channel Receptor Agonist

| | GAG release | |
|---|---|---|
| | Mean ± SEM | |
| Treatment | (μg/explant) | % Inhibition |
| control* | 13 ± 1.22 | — |
| IL-1 (100 ng/ml)** | 61 ± 7.16 | 0 |
| IL-1 + Formula IIc (0.01 μM) | 62 ± 4.82 | −2 |
| IL-1 + Formula IIc (0.03 μM) | 59 ± 4.62 | 4 |
| IL-1 + Formula IIc (0.10 μM) | 43 ± 3.18 | 38 |
| IL-1 + Formula IIc (1.00 μM) | 14 ± 1.44 | 98 |

*cartilage explant no IL-1 and no agonist
**IL-1 alone with no agonist

### Example 13

### Reduction of Aggrecan Degradation in Human Cartilage Explants

To demonstrate efficacy of TRPV4 channel receptor agonists in blocking aggrecan degradation in human cartilage, explants were prepared from normal articular cartilage derived from a 32 year old donor, and the effect of several TRPV4 channel receptor agonists, including Formula IId and Formula Ib, were tested for their ability to inhibit IL-1-induced aggrecan degradation. Because the progression of aggrecan degradation in response to IL-1 in human cartilage is slower than in bovine cartilage, culture media was removed and replaced with fresh treatments every 2-3 days for approximately 3 weeks. For each timepoint, GAG levels were measured by DMMB assay, and cumulative data released over the entire 3 week culture period was calculated.

Unlike cartilage explants derived from other species, human cartilage undergoes a proteolytically-driven aggrecan degradation in the absence of IL-1 stimulation, presumably by cartilage destruction due to an undiagnosed arthritic condition. While the identity of the catabolic stimulus in control cultures is unknown, an endogenous activator may play a pivotal role in inducing cartilage destruction in vivo. This example demonstrates that aggrecan degradation of unstimulated human cartilage explants can be blocked by SB703704, a selective aggrecanase inhibitor [Yao, et al. (2001) J. Med. Chem. 44, 3347-3350], suggesting the contribution of aggrecanase.

To address whether TRPV4 channel receptor agonists are equally effective in blocking aggrecan degradation in unstimulated and IL-1-stimulated human cartilage, compounds were evaluated using a 3-point concentration response both in the absence and in the presence of IL-1. Table 6 shows the effect of Formula IId on aggrecan degradation as a representative observation for other TRPV4 channel receptor agonists tested. The data represents cumulative aggrecan degradation after 21 days in culture. In the absence of IL-1, Formula IId, as well as other TRPV4 channel receptor agonists, was effective in blocking aggrecan degradation, suggesting that TRPV4 channel receptor agonists will be effective in blocking degradation induced by an endogenous catabolic stimulus present in human cartilage. In addition, Formula IId caused a concentration-dependent reduction of IL-1-induced aggrecan degradation. Consistent with TRPV4 channel receptor agonists being effective in blocking aggrecan degradation induced by both an endogenous activator in control cartilage, as well as IL-1-stimulated degradation, the level of inhibition at the highest concentration of formula IId was greater than 100%. The estimated IC50's shown in Table 7 are very similar to those IC50's calculated from IL-1-stimulated bovine cartilage. Based on a high degree of homology between human and bovine TRPV4 channel receptor (−95%), and that a number of key TRPV4 channel receptor agonists are equally potent in blocking aggrecan degradation induced in bovine and human cartilage explants, these data support the hypothesis that bovine cartilage is a suitable tissue to use to predict efficacy of TRPV4 channel receptor agonist activity in human cartilage.

Further, the effective concentration of Formula IId to inhibit IL-1-induced aggrecan degradation is similar to the concentrations of compound used to demonstrate calcium influx by electrophysiology, demonstrating a correlation exists between activities by Formula IId to induce calcium influx and to inhibit aggrecan degradation.

TABLE 6
Formula IId is Effective in Inihbiting Aggrecan Degradation Both in
Unstimulated and IL-1-Stimulated Human Cartilage Explants.

| | | | | GAG | |
|---|---|---|---|---|---|
| | | | | Mean ± SEM | % |
| Control/IL-1 | compound | conc. (uM) | | (ug/mg) | inhibition |
| control | none | 0 | | 6.7 ± 0.3 | 0 |
| control | IId | 0.01 | uM | 5.9 ± 0.3 | 12* |
| control | IId | 0.1 | uM | 4.4 ± 0.2 | 34* |
| control | IId | 1 | uM | 3.7 ± 0.3 | 45* |
| IL-1 (100 ng/ml) | none | 0 | | 20.8 ± 1.8 | 0 |
| IL-1 (100 ng/ml) | IId | 0.01 | uM | 15.9 ± 1.6 | 35** |
| IL-1 (100 ng/ml) | IId | 0.1 | uM | 8.2 ± 0.5 | 89** |
| IL-1 (100 ng/ml) | IId | 1 | uM | 4.8 ± 0.5 | 113** |

*relative to control GAG release;
**relative to IL-1 stim. GAG release

TABLE 7
Effect of TRPV4 Channel Receptor Compounds on Chondrocyte-Based
Functional Assays

| | | | | | | Aggrecan | |
|---|---|---|---|---|---|---|---|
| | Ca | | Ca | | | Degradation | |
| | Influx - | | Influx - | | | Cartilage | |
| | FLEX | | FLEX | | | Explants (IC50) | |
| | (bovine) | | (human) | | Aggrecanase | | human |
| Com- | EC50 | % | EC50 | % | Production | bovine | IC50 |
| pound | (uM) | effic. | (uM) | effic. | IC50 (uM) | IC50 (uM) | (uM) |
| IId | 0.12 | 79 | 0.11 | 179 | 0.04 | 0.01 | 0.03 |
| Ib | 0.28 | 119 | 0.26 | 133 | 0.6 | 0.2 | 0.3 |
| IIIb | 0.29 | 70 | 0.28 | 78 | 0.35 | 0.4 | 0.5 |

Additional studies have demonstrated the efficacy of TRPV4 channel receptor agonists referenced above in arresting further GAG release in cartilage explants actively undergoing matrix degradation, further demonstrating the importance of TRPV4 as an important regulatory protein in chondrocytes.

### Example 14

### Reduction of IL-1 Induced Collagen Degradation (Bovine Articular Cartilage Explants)

IL-1 stimulates collagen degradation in bovine articular cartilage explants after extended (−2-3 weeks) exposure to IL-1. Collagen breakdown was evaluated by measuring the levels of hydroxyproline, a major component of hydrolyzed collagen fibrils, using a colorimetric assay that is understood in the art. Bovine articular cartilage was stimulated with IL-1 for 18 days, in the absence or presence of Formula Ib or Formula IId (both at 1 uM). Media was removed and replaced every 2-4 days with fresh IL-1 and compound through day 18. As has been shown previously, aggrecan degradation precedes collagen breakdown in IL-1-stimulated bovine cartilage explants. Once aggrecan depletion had been achieved by day 10, media were analyzed for collagen breakdown using the hydroxyproline assay. Collagen degradation in conditioned media generated from day 14-18 was evaluated and is presented in Table 8. IL-1 caused a significant level of collagen degradation, and this effect was completely blocked by Formula Ib (1 uM), and partially, but effectively, blocked by Formula IId. In separate studies, broad spectrum MMP inhibitors (for example CGS-27023A—Ganu, et al., Ann NY Acad Sci. 1999; 878:607-11) completely blocked IL-1-induced collagen degradation, supporting the hypothesis that MMP's contribute to the collagen degradation in this model. Together, these data are consistent with TRPV4 channel receptor agonists blocking production of metalloproteinases, including MMP-13, induced by IL-1 that contribute to collagen degradation. These data also confirm that TRPV4 channel receptor agonists provide protection to both major cartilage matrix components, aggrecan and type II collagen.

TABLE 8
TRPV4 Channel Receptor Agonists Inhibit Collagen Degradation
Induced by IL-1

| | | | Hydroxyproline | % |
|---|---|---|---|---|
| Treatment | Concentration | | Mean ± SEM (ug) | inhibition |
| control | | | 2 ± 0.2 | — |
| IL-1 | 50 | ng/ml | 75 ± 10 | 0 |
| IL-1 + Ib | 1 | uM | 6 ± 2 | 95 |
| IL-1 + IIb | 1 | uM | 16 ± 6 | 81 |

### Example 15

### TRPV4 Channel Receptor Agonist Decreases Nitric Oxide Levels

Nitric oxide (NO) is a free radical that is believed to contribute to the tissue destruction in osteoarthritis. While having direct effects on cartilage matrix damage, NO can also contribute by activating latent proteases which leads to increased tissue destruction. To evaluate the effect of TRPV4 channel receptor agonists on NO production, bovine articular chondrocytes were embedded in alginate beads as described in Hauselmann, et al. (1992) Matrix 12: 116-129, and NO levels were determined in the conditioned media by Greiss reaction (Badger A M, et al. (1998) J. Immunol. 161: 467-473), following stimulation by IL-1, in the absence or presence of TRPV4 channel receptor agonist. IL-1 has been shown to induce NO production in chondrocytes, and Formula IIb caused a concentration dependent reduction in IL-1-induced NO production as shown in Table 9.

TABLE 9
Reduction of IL-1 Induced Nitric Oxide Release (Mean ± SE) by a
TRPV4 Channel Receptor Agonist in bovine articular chondrocytes
in alginate beads.

| | NO levels | % |
|---|---|---|
| Treatment | Mean ± SEM (μM) | inhibition |
| Control* | 1 ± 0 | — |
| IL-1 (10 ng/mL)** | 27 ± 1 | 0 |
| IL-1 + Formula IIb (0.3 μM) | 17 ± 1 | 38 |
| IL-1 + Formula IIb (1.0 μM) | 6 ± 1 | 81 |
| IL-1 + Formula IIb (3.0 μM) | 2 ± 0 | 96 |

*no IL-1 or agonist
**IL-1 alone no agonist

### Example 16

### Attenuation of IL-1 Induced Inhibition of Proteoglycan Synthesis

Although cartilage degradation is the hallmark of osteoarthritis, there is also a reduction in the ability of chondrocytes to resynthesize damaged matrix. It has been observed that cytokines, including IL-1, cause a significant reduction in collagen and proteoglycan (PG) synthesis. The effect on matrix synthesis, combined with the stimulation of matrix breakdown by these cytokines, leads to an overall net loss of cartilage matrix. The effects of TRPV4 channel receptor agonists on the synthesis of proteoglycans was measured by [³⁵S] incorporation in bovine articular chondrocytes embedded in alginate beads. IL-1 caused an inhibition of [³⁵S] incorporation into large molecular weight proteoglycan monomers, and Formula IIb caused a concentration-dependent reversal of the IL1-mediated inhibition of PG synthesis as shown in Table 10. These data suggest that a TRPV4 channel receptor agonist can attenuate the inhibition of matrix synthesis observed in OA.

TABLE 10
Attenuation of IL-1 Induced Inhibition of PG Synthesis (Mean ± SE)
by a TRPV4 Channel Receptor agonist in Bovine Articular Chondrocytes
in Alginate Beads.

| | PG synthesis | % IL-1 |
|---|---|---|
| Treatment | Mean ± SEM (cpm/bead) | reversal |
| control* | 2413 ± 93 | — |
| IL-1 (10 ng/mL)** | 825 ± 49 | 0 |
| IL-1 + Formula IIb (0.3 uM) | 878 ± 48 | 3 |
| IL-1 + Formula IIb (1.0 uM) | 1066 ± 69 | 15 |
| IL-1 + Formula IIb (3.0 uM) | 1530 ± 73 | 44 |

*no IL-1 or agonist
**IL-1 alone no agonist

### Example 17

### TRPV4 Channel Receptor Electrophysiology

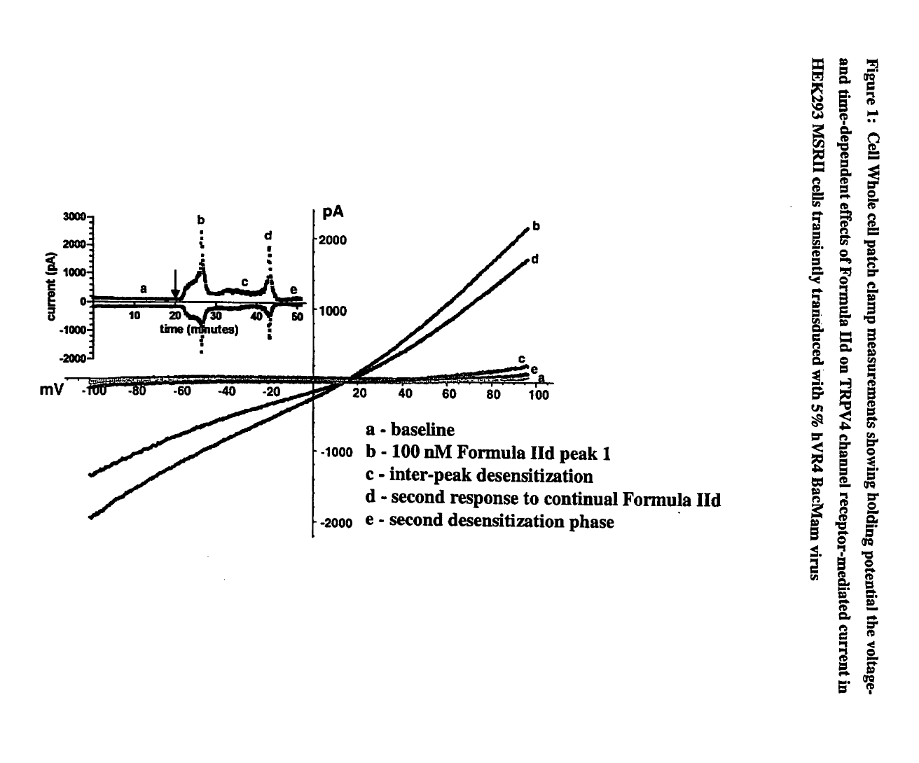

Functional, cell-based assays presently utilized to evaluate the activity of TRPV4 channel receptor agonists (e.g., FLIPR) provide an indirect, downstream measure of channel activation or blockage. In order to more directly evaluate a compound's effect on TRPV4 channel receptor, procedures to electrophysiologically record current flow through TRPV4 channels have been established. These procedures utilize HEK293 MSRII cells transiently transduced with 5% human TRPV4 BacMam virus, a standard extracellular physiological saline solution, and a well established, potassium channel-blocking electrode (internal) solution (Vriens et al., PNAS, 2004). Standard whole cell patch clamp techniques were used to maintain cells at a holding potential of 0 mV between inductions of 200 msec, −100 mV to +100 mV ramp protocols every 5 seconds. A representative example of the effects of a TRPV4 channel receptor agonist, Formula IId, is shown in FIG. 1. The inset of FIG. 1 represents the inward and outward current amplitude at −100 mV and +100 mV, respectively, during each ramp protocol prior to (baseline) and throughout the administration of 100 nM Formula IId. The main body of FIG. 1 illustrates the recorded current flow in response to a −100 mV to +100 mV ramp at the five specific times labelled in the inset. Formula IId activated a human TRPV4 channel receptor in a complex manner that involved an initial activation phase, rapid desensitization and, in this but not all cases, a second activation/desensitization phase. Concentrations as low as 10 nM have been observed to activate the human TRPV4 channel receptor. In separate experiments, 3 μM Formula IId had no measurable effect on membrane currents in HEK293 MSRII cells exposed to 5% BacMam virus alone (i.e., without human TRPV4 vector) as shown in FIG. 1.

### Example 18

### EC50 Values for Human (HAC), Bovine (BAC), and rat (RAC) Articular Chondrocytes for Acyclic 1,3-diamines

EC50 values for compound Formulas IIIb and Formula IIIc were determined using a FLIPR assay and FlexStation (manufactured by Molecular Devices (Sunnyvale, Calif.)) for Ca²⁺ influx in bovine, rat and human articular chondrocytes. In addition, IC50 values were determined for these molecules using an aggrecanase production assay using techniques described in Examples 11 and 13, described above. EC50 and IC50 values of compounds IIIb and IIIc are presented below in Table 11.

TABLE 11
Effect of TRPV4 channel receptor agonists on FLIPR, Explants and
Functional Assays

| | | | RAC | HAC | |
|---|---|---|---|---|---|
| | FLIPR | BAC | EC50 uM | EC50 uM | Aggrecanase |
| Com- | EC50 | EC50 uM | (% eff) | (% eff) | Production |
| pound | (uM) | (% eff) | N = 2 | N = 2 | IC50 uM |
| IIIb | 0.7 | 0.26 | 0.7 (104%) | 0.21 (130%) | 0.4 |
| | | (104%) | 0.37 (99%) | 0.24 (124%) | |
| IIIc | 0.37 | 0.31 | 0.14 (70%) | 0.25 (127%) | 0.04 |
| | | (94%) | | | |

The above description fully discloses how to make and use the present invention. However, this invention is not limited to the particular embodiments described hereinabove, but includes all modification thereof within the scope of the appended claims and their equivalents. Those skilled in the art will recognize through routine experimentation that various changes and modifications can be made without departing from the scope of this invention.

1. A method for activating a TRPV4 channel receptor or a TRPV4 channel receptor variant in at least one cell expressing the TRPV4 channel receptor or TRPV4 channel receptor variant comprising the step of: contacting said at least one cell with an effective amount of pharmaceutical composition comprising an agonist to the said TRPV4 channel receptor.

2. The method of claim 1, wherein said at least one cell is from a human.

3. The method of claim 1, wherein said at least one cell is a chondrocyte.

4. The method of claim 1, wherein said at least one cell is part of a cartilage matrix.

5. The method of claim 1, wherein the agonist reduces an amount of at least one type of matrix degrading enzymes produced by said at least one cell.

6. The method of claim 1, wherein the agonist reduces an amount of at least one type of matrix degrading enzymes released by said at least one cell.

7. The method of claim 1, wherein the agonist reduces the amount of aggrecanase produced by said at least one cell.

8. The method of claim 1, wherein the agonist reduces the amount of aggrecanase released by said at least one cell.

9. The method of claim 1, wherein the agonist reduces an amount of at least one type of matrix metalloprotease produced by said at least one cell.

10. The method of claim 1, wherein the agonist reduces an amount of at least one type of matrix metalloprotease MMPs released by said at least one cell.

11. The method of claim 9, wherein at least one matrix metalloprotease is chosen from the group of: MMP-1, MMP-3 and MMP-13.

12. The method of claim 1, wherein the agonist reduces the amount of nitric oxide produced by said at least one cell.

13. The method of claim 1, wherein the agonist reduces the amount of nitric oxide released by said at least one cell.

14. The method of claim 1, wherein the agonist attenuates inhibition of proteoglycan synthesis.

15-27. (canceled)

28. The method of claim 1, wherein the agonist increases current flow through said TRPV4 channel receptor.

29. A method for treating a patient in need thereof comprising contacting at least one cell expressing a TRPV4 channel receptor of the patient with a therapeutically effective amount of an agonist to the TRPV4 channel receptor.

30. The method of claim 29, wherein the patient is suffering from a disease of the cartilage.

31. The method of claim 29, wherein the patient is suffering from a disease or condition chosen from the group of: pain, chronic pain, neuropathic pain, postoperative pain, osteoarthritis, neuralgia, neuropathies, algesia, nerve injury, ischaemia, neurodegeneration, inflammatory diseases and cartilage degeneration.

32. The method of claim 29, wherein the patient suffers from a diseases affecting the larynx, trachea, auditory canal, intervertebral discs, ligaments, tendons, joint capsules or bone development.

33. The method of claim 29, wherein the disease is related to joint destruction.

34. The method of claim 33, wherein the patient is suffering from osteoarthritis.

35. The method of claim 33, wherein the patient is suffering from rheumatoid arthritis.

36-38. (canceled)

39. The method of claim 29, further comprising reducing the amount of aggrecan degradation in the patient.

40. The method of claim 29, further comprising reducing the amount of collagen degradation in the patient.

41. The method of claim 29, further comprising attenuating cartilage degradation in the patient in response to inflammatory mediators.

42. The method of claim 29, further comprising attenuating cartilage degradation in the patient in response to injury.

43. The method of claim 29, further comprising attenuating decreased matrix protein production in the patient.

44. The method of claim 43, wherein the matrix protein is chosen from the group of: aggrecan, type II collagen and type VI collagen.

45. The method of claim 29, further comprising attenuating increased production of matrix degrading enzymes.

46. The method of claim 45, wherein the matrix degrading enzymes are chosen from the group of: MMP-1, MMP-3, MMP-9, MMP-13, ADAMTS4, and ADAMTS5.

47. The method of claim 46, wherein the production of matrix degrading enzymes are induced by inflammatory mediators.

48. The method of claim 41, wherein the production of matrix degrading enzymes are induced due to injury.

49. The method of claim 29, further comprising reducing the amount of nitric oxide produced by at least one cell in cartilage.

50. The method of claim 29, further comprising attenuating inhibition of proteoglycan synthesis.

51. A compound comprising a 1,3-diamine wherein said compound activates a TRPV4 channel receptor or a TRPV4 channel receptor variant when said compound is contacted with at least one cell expressing said TRPV4 channel receptor or said TRPV4 channel receptor variant.

52-63. (canceled)
